SEQUENCE LISTING (2 sequences)

SEQ ID NO: 1 - protein, 564 aa, Rhodococcus jostii
MTTSMKAANPMNFPSTSDTGIVDVLGVGAGFSGLYLSHRLTTAGWTFAGFEAGPSVGGTWFWNTYPGARCDVESIYYSYSFDEALQQEWTWSQRFAPQAEILSYINHVADRFDLRKHFTFNTRVVGATWNAAERLWEVQLDNGETRRGRYLISGAGGLSTPKDFDVPGLGNFTGLQVSTSRWNISLDDLAGKRVAVIGTGSSGVQAIPLIAEVAEHVTVFQRTPNYVMPARNAELPLERVDSIKDDYPAIREECRHSPGGIPDRPVTDKAFDVSAEERQRRYEAAYERSGFNGVGGEFADLLTDVEANRTASEFIHDKIREIVEDPATAELLVPRYHPLGAKRSVFGTDYYETYNRPNVSLVSLRDEPIETMTANAIVTSKGTYEADAVVLAIGFDAFTGPLYGLGLTGASGRKLQETWQDGIRTYLGMMTTDFPNFFMVAGPQSPALASNVVMTIEQAVDWIADLIEHARDSGATLVEATPEGQNDWVDITEETVAQTLYATTDSWYRGSNVEGKPNTFMGYVGGVGKYRRMCTEIAKRGYPGVRIDGETESPHLGPIHREIS

SEQ ID NO: 2 - protein, 352 aa, Thermoanaerobacter brockii
MKGFAMLSIGKVGWIEKEKPAPGPFDAIVRPLAVAPCTSDIHTVFEGAIGERHNMILGHEAVGEVVEVGSEVKDFKPGDRVVVPAITPDWRTSEVQRGYHQHSGGMLAGWKFSNVKDGVFGEFFHVNDADMNLAHLPKEIPLEAAVMIPDMMTTGFHGAELADIELGATVAVLGIGPVGLMAVAGAKLRGAGRIIAVGSRPVCVDAAKYYGATDIVNYKDGPIESQIMNLTEGKGVDAAIIAGGNADIMATAVKIVKPGGTIANVNYFGEGEVLPVPRLEWGCGMAHKTIKGGLCPGGRLRMERLIDLVFYKRVDPSKLVTHVFRGFDNIEKAFMLMKDKPKDLIKPVVILA

(19) United States
(12) Patent Application Publication
(10) Pub. No.: US 2023/0407352 A1
(43) Pub. Date: Dec. 21, 2023

(54) Method for the Production of Thiocarbamate Derivatives A2AR Inhibitors

(71) Applicant: iTeos Belgium SA, Gosselies (BE)

(72) Inventors: Xuecheng Jiao, Tianjin (CN); Michael Deligny, Rhode Saint Genese (BE); Stefano Crosignani, Vevey (CH); Xiangjun Jiang, Tianjin (CN)

(73) Assignee: iTeos Belgium SA, Gosselies (BE)

(21) Appl. No.: 18/034,449

(22) PCT Filed: Oct. 29, 2021
(86) PCT No.: PCT/CN2021/127308

§ 371 Date: Apr. 28, 2023

Related U.S. Application Data
(63) Continuation of application No. PCT/CN2020/125266, filed on Oct. 30, 2020.

(51) Int. Cl. C12P 17/12; C07D 513/14; C12N 9/02; C12N 9/04

(52) U.S. Cl. CPC C12P 17/12; C12N 9/0006; C12N 9/0071; C07D 513/14

(57) ABSTRACT

The present disclosure relates to synthesis of enantiomerically rich key drug intermediates as a means for manufacturing of thiocarbamate derivatives as A2A adenosine receptor (A2AR) inhibitors. More particularly, the present disclosure provides a viable efficient technology using enzymatic biotransformation process which utilizes cheaper substrate for production of high value key intermediates for A2AR inhibitors.

### FIELD

The present disclosure relates to synthesis of enantiomerically rich key drug intermediates as a means for manufacturing of thiocarbamate derivatives. More particularly, the present disclosure relates to the use of enzymatic biotransformation process in the manufacture of thiocarbamate derivatives which are useful as A2A adenosine receptor (A2AR) inhibitors.

### BACKGROUND

Chirality plays an essential role in drugs. The pharmacological activity of drugs depends mainly on its interaction with biological targets such as proteins, nucleic acids and bio membranes. Most biological molecules (proteins, sugars, etc.) are present in only one of many chiral forms, so different enantiomers of a chiral drug molecule bind differently (or not at all) to target receptors. One enantiomer of a drug may have a desired beneficial effect while the other may cause serious and undesired side effects, or sometimes even beneficial but entirely different effects.

Biological activity of the two enantiomers of a chiral drug or chiral key drug intermediates has raised the demand for enantiomerically pure products. The production of chirality with maximum economy is one of the most challenging tasks of today's pharmaceutical industry. One way to obtain pure enantiomers is the separation of racemates via kinetic resolution through preferred crystallization or preparative chromatography such as simulated moving bed (SMB) chromatography on chiral stationary phases. Simulated moving-bed chromatography can be used for the separation of the two enantiomers of a chiral molecule, which is feasible at all production scales, from laboratory to pilot to production plant. However, simulated moving-bed enantiomer separation can make the development process of a new chiral drug or drug intermediate substantially costly, longer and less efficient (low yield).

The rapid progress in biocatalysis in the identification and development of enzymes over the last decade has enormously enlarged the chemical reaction space that can be addressed not only in research applications, but also on industrial scale.

The Applicant provided a series of A2AR inhibitors in international patent application PCT/EP2018/058301, being thiocarbamate derivatives, which are useful to restore immune functions in tumor environment. Consequently, there remains a need for an efficient, cost-effective process for the production of these compounds in high yield. The present disclosure provides a viable efficient technology using cell-free biosynthesis system which utilizes cheaper substrate for production of high value key intermediates for A2AR inhibitors catalyzed by highly efficient enzymes.

### SUMMARY

Provided herein is a process for preparing (+)-1-(2,4-difluoro-5-(2-(methylsulfinyl)ethoxy)phenyl)piperazine (Intermediate B), or a pharmaceutically acceptable salt or solvate thereof,

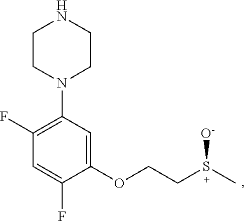

comprising the step of contacting 1-(2,4-difluoro-5-(2-(methylthio)ethoxy)phenyl)piperazine, or a pharmaceutically acceptable salt thereof:

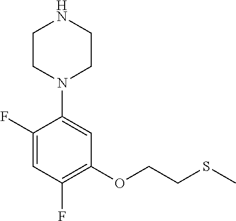 [SMILES: CSCCOc1cc(N2CCNCC2)c(F)cc1F]

with an enzyme in a solvent.

Additionally, provided herein is an enzymatic biotransformation process for manufacturing compound of Formula (B) or a pharmaceutically acceptable salt or solvate thereof, wherein Formula (B) is at least 80%, at least 90%, at least 95%, at least 99%, at least 99.9% enantiomerically pure, wherein the enzymatic biotransformation comprises a solvent, and the temperature of the biotransformation is below the boiling temperature of the solvent

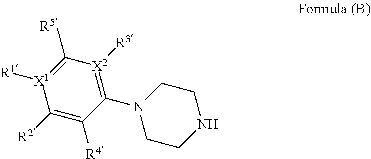

wherein

X¹ and X² represent each independently C or N; R1 is absent when X1 is N; or when X¹ is C, R¹′ represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino or alkylsulfonealkyl; said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylamino alkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;

R²′ represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino, or alkylsulfonealkyl; said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)amino carbonyl, alkylamino alkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;

or R¹′ and R²′ form together with the atoms to which they are attached a 5- or 6-membered aryl ring, a 5- or 6-membered heretoaryl ring, a 5- or 6-membered cycloalkyl ring or a 5- or 6-membered heretocyclyl ring; optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alky loxy carbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl; wherein each of R^{1′} and R^{2′} comprises at least one sulfoxide as permitted by valency;

R³′ is absent when X2 is N; or when X2 is C, R3 represents H or halo, preferably H or F;

R⁴′ represents H or halo, preferably H or F; and R5′ represents H or halo, preferably H or F.

Provided herein, an enzymatic biotransformation process for manufacturing the compound of formula (B-1) or a pharmaceutically acceptable salt or solvate thereof,

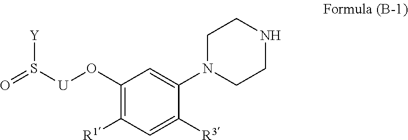

wherein

Formula (B) is at least 80%, at least 90%, at least 95%, at least 99%, at least 99.9% enantiomerically pure;

R1′ and R3′ are as defined above in this disclosure;

U represents alkylene, arylene, heteroarylene or heterocyclyene optionally substituted by one or more substituent selected from halo, hydroxy, alkyl, heterocyclylalkyl, hydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkoxy, amino, dialkylamino, aminoalkylcarbonylamino, aminocarbonylalkylamino, heterocyclylalkylaminocarbonyl, (aminocarbonylalkyl)(alkyl)amino, hydroxycarbonyl, aminocarbonyl, aminoalkylaminocarbonyl, alkylaminoalkylaminocarbonyl, dialkylaminoalkylaminocarbonyl, (alkylaminoalkyl)(alkyl)aminocarbonyl, heterocyclylcarbonyl, alkylsulfoxide and alkylsulfonealkyl;

Y represents represents an alkyl, arylene, heteroarylene or heterocyclyene optionally substituted by one or more group selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;

or U and Y form together with the atoms to which they are attached an aryl ring, a heteroaryl ring, a cycloalkyl ring or a heterocyclyl ring; optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In one aspect, the compound of Formula (B) is selected from the group consisting of:
(S)-1-(2-fluoro-4-(2-(methylsulfinyl)ethoxy)phenyl)piperazine;
(R)-1-(2-fluoro-4-(2-(methylsulfinyl)ethoxy)phenyl)piperazine;
(S)-1-(2,4-difluoro-5-(2-(methylsulfinyl)ethoxy)phenyl)piperazine; and
(R)-1-(2,4-difluoro-5-(2-(methylsulfinyl)ethoxy)phenyl)piperazine.

Provided herein, a process for manufacturing a compound of Formula (Ia), or a pharmaceutically acceptable salt or solvate thereof,

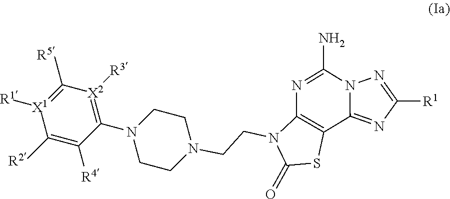

wherein

R¹ represents 5- or 6-membered heteroaryl or 5- or 6-membered aryl, wherein heteroaryl or aryl groups are optionally substituted by one or more substituent selected from C1-C6 alkyl (preferably methyl) and halo (preferably fluoro or chloro); preferably R1 represents 5-membered heteroaryl; more preferably R1 represents furyl;

X¹ and X² represent each independently C or N; R1 is absent when X1 is N; or when X1 is C, R1′ represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino or alkylsulfonealkyl; said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylamino alkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;

R2′ represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino, or alkylsulfonealkyl; said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)amino carbonyl, alkylamino alkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl; or R1′ and R2′ form together with the atoms to which they are attached a 5- or 6- membered aryl ring, a 5- or 6-membered heretoaryl ring, a 5- or 6-membered cycloalkyl ring or a 5- or 6-membered heretocyclyl ring; optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alky loxy carbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;

wherein each of R^{1′} and R^{2′} comprises at least one sulfoxide as permitted by valency;

R3′ is absent when X2 is N; or when X2 is C, R3 represents H or halo, preferably H or F;

R4′ represents H or halo, preferably H or F; and R5′ represents H or halo, preferably H or F,

In one aspect, the process for manufacturing a compound of Formula (Ia), or a pharmaceutically acceptable salt or solvate thereof comprises the steps of:
synthesis of diamine intermediate of Formula (B) by an enzymatic biotransformation, wherein the synthesis produces the intermediate of Formula (B) at least 80%, at least 90%, at least 95%, at least 99%, or at least 99.9% enantiomerically purity:

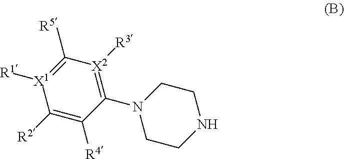

wherein, R^{1′}, R^{2′}, R^{3′}, R^{4′}, and R^{5′} are as defined above in this claim, the enzymatic biotransformation is performed in a solvent, and the temperature of the biotransformation is below the boiling temperature of the solvent;
introduction of an intermediate of Formula (A) and the intermediate of Formula (B) into a suitable solvent:

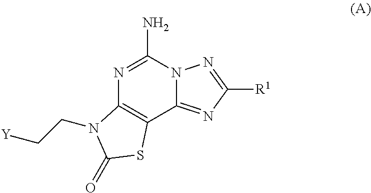

wherein, R¹ is as defined above in this claim and Y represents halo, alkylsulfonyloxy having 1 to 6 carbon atoms or arylsulfonyloxy having 6 to 10 carbon atoms; and
coupling between intermediate of Formula (B) and intermediate of Formula (A) in the suitable solvent at a temperature ranging from about 20° C. to about 180° C., thereby forming the compound of Formula (Ia).

In one aspect, the intermediate of Formula (B) is selected from the group consisting of:
(S)-1-(2-fluoro-4-(2-(methylsulfinyl)ethoxy)phenyl)piperazine;
(R)-1-(2-fluoro-4-(2-(methylsulfinyl)ethoxy)phenyl)piperazine;
(S)-1-(2,4-difluoro-5-(2-(methylsulfinyl)ethoxy)phenyl)piperazine; and
(R)-1-(2,4-difluoro-5-(2-(methylsulfinyl)ethoxy)phenyl)piperazine.

In one aspect, the compound of Formula (Ia) is selected from the group consisting of: (S)-5-amino-3-(2-(4-(2-fluoro-4-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2-fluoro-4-(2-(methylsulfinyl)ethoxy)phenyl)-piperazin-1-yl)ethyl)-8-(furan-2-yl)thiazolo[5,4-e][1,2,4]triazolo[1,5-c]pyrimidin-2(3H)-one;
(+)-(S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(−)-(R)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(S)-5-amino-8-(furan-2-yl)-3-(2-(4-(4-(methylsulfinyl)phenyl)piperazin-1-yl)ethyl)thiazolo[5,4-e][1,2,4]triazolo[1,5-c]pyrimidin-2(3H)-one;
(R)-5-amino-8-(furan-2-yl)-3-(2-(4-(4-(methylsulfinyl)phenyl)piperazin-1-yl)ethyl)thiazolo[5,4-e][1,2,4]triazolo[1,5-c]pyrimidin-2(3H)-one;
(S)-5-amino-3-(2-(4-(2,4-difluoro-5-(methylsulfinyl)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2,4-difluoro-5-(methylsulfinyl)phenyl)piperazin-1-yl)ethyl)-8-(furan-2-yl)thiazolo[5,4-e][1,2,4]triazolo[1,5-c]pyrimidin-2(3H)-one;
(S)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-(2-(methylsulfinyl)ethyl)benzamide;
(R)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-(2-(methylsulfinyl)ethyl)benzamide;
(S)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-methyl-N-(2-(methylsulfinyl)ethyl)benzamide;
(R)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-methyl-N-(2-(methylsulfinyl)ethyl)benzamide;
(R)-5-amino-3-(2-(4-(2-fluoro-4-(methylsulfinyl)phenyl)piperazin-1-yl)ethyl)-8-(furan-2-yl)thiazolo[5,4-e][1,2,4]triazolo[1,5-c]pyrimidin-2(3H)-one;
(S)-5-amino-3-(2-(4-(2-fluoro-4-(methylsulfinyl)phenyl)piperazin-1-yl)ethyl)-8-(furan-2-yl)thiazolo[5,4-e][1,2,4]triazolo[1,5-c]pyrimidin-2(3H)-one;
(S)-4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methylsulfinyl)ethyl)benzamide;
(R)-4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methylsulfinyl)ethyl)benzamide;
(S)-5-amino-3-(2-(4-(2,4-difluoro-5-(3-(methylsulfinyl)propoxy)phenyl)piperazin-1-yl)ethyl)-8-(furan-2-yl)thiazolo[5,4-e][1,2,4]triazolo[1,5-c]pyrimidin-2(3H)-one; and pharmaceutically acceptable salts or solvates thereof.

In some embodiments, the enzymatic biotransformation is in a cell free system.

In some embodiments, the enzymatic biotransformation comprises at least one enzyme selected from oxidoreductases.

In some embodiments, the oxidoreductases are selected from the group of monooxygenases and/or alcohol dehydrogenases.

In a preferred embodiment, the monooxygenase is derived from Rhodococcus jostii.

In a preferred embodiment, the monooxygenase comprises a polypeptide sequence having at least 85%, 86%, 87%, 88%, 89%, 90%, 91%, 92%, 93%, 94%, 95%, 96%, 97%, 98%, 99%, or more sequence identity to SEQ ID NO:1.

In a preferred embodiment, the alcohol dehydrogenase comprises a polypeptide sequence having at least 85%, 86%, 87%, 88%, 89%, 90%, 91%, 92%, 93%, 94%, 95%, 96%, 97%, 98%, 99%, or more sequence identity to SEQ ID NO: 2.

In a preferred embodiment, the alcohol dehydrogenase is derived from Thermoanaerobium brockii.

In some embodiments, the enzymes are in crude or purified or in immobilized form.

In some embodiments, the temperature in step (i) is between about 0° C. and about 45° C.

In some embodiments, the solvent in step (i) comprises water and/or isopropanol.

In some embodiments, the step (i) further comprises nicotinamide adenine dinucleotide phosphate (NADP+).

In some embodiments, the steps (i), (ii) and (iii) are performed in a reactor. In some embodiments, the reactor is different for each step (i), (ii) and (iii).

In some embodiments, enantiomerically pure compound of Formula (B) is ‘R’ isomer.

In some embodiments, enantiomerically pure compound of Formula (B) is ‘S’ isomer.

In some embodiments, the synthesis of intermediate of Formula (B) comprises a reaction yield. In one embodiment, the reaction yield is at least about 50%.

In one aspect, the synthesis of intermediate of Formula (B) comprises overoxidation of an undesired enantiomer, whereby undesired enantiomer is converted to a sulfone.

In some embodiments, the sulfone is removed by recrystallization.

In one aspect, provided herein is a process for the synthesis of a compound of formula (IV)

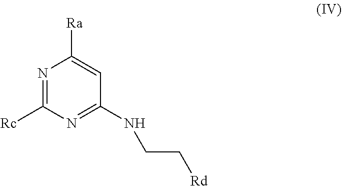

wherein, Ra is absent or is halo; Rb is halo; Rc is absent or is amino; Rd is absent or is methoxy; comprising the steps of: reacting the compound of formula (II)

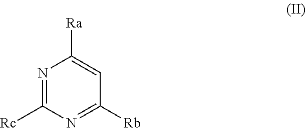

with a compound of formula (III)

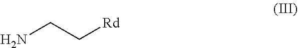

in the presence of a suitable base.

In some embodiments, the base is an organic base. In some embodiments, the organic base is selected from an acyclic amine (e.g., triethylamine, diisopropylethylamine (DIPEA), etc.) or a cyclic amine (e.g., pyrrolidine, piperidine, etc.).

In some embodiments, Ra is chlorine; Rb is chlorine; Rc is amino and Rd is methoxy.

Provided herein is a process for the synthesis of a compound of formula (V)

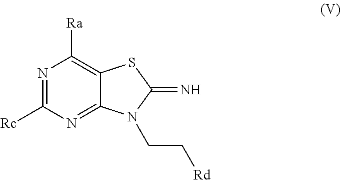

wherein, Ra is absent or is halo; Rc is absent or is amino; Rd is absent or is methoxy; comprising the steps of: (i) preparing the compound of formula (IV) according to process described above in this disclosure; (ii) reacting compound of formula (IV) with KSCN and Br₂ in the presence of an acid at a temperature below 0° C.; and (iii) addition of a base selected from ammonia, barium hydroxide, calcium hydroxide, cesium hydroxide, magnesium hydroxide, potassium hydroxide, or sodium hydroxide to yield a compound of formula (V).

The base can be made available in the form of a resin (such as Amberlite® and the like). In some further embodiments, the base can be provided in the form of a solution in water such as about 2N solution (e.g., about 0.5N solution, about 1N solution, about 1.5N solution, about 2.5N solution, from about 3N to about 5N solution, from about 5N to about 10N solution).

In some embodiments, the step (ii) can be performed at a temperature from about from about −10° C. to about 0° C., from about −5° C. to about −15° C., from about −10° C. to about −25° C., or from about −15° C. to about −35° C.

Provided herein is a process for the synthesis of a compound of formula (VI)

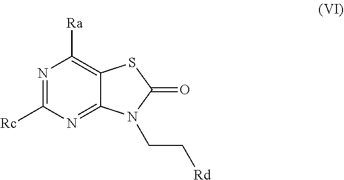

wherein, Ra is absent or is halo; Rc is absent or is amino; Rd is absent or is methoxy; comprising the steps of: (i) preparing the compound of formula (IV) according to process described above in this disclosure; (ii) dissolving the compound of formula (IV) in a water miscible organic solvent. (iii) addition of an acid to yield a compound of formula (VI).

In some embodiments, water miscible organic solvent can be selected from dioxane, THF, water, acetic acid, DME, DMF, DMSO, acetonitrile, diglyme, alcohol (e.g. methanol, ethanol or tert-butanol) methyl-ethyl ketone, NMP, or mixtures thereof.

In some embodiments, the acid can be selected from TFA, hydrochloric acid, hydrobromic acid, sulfuric acid, nitric acid, phosphoric acid, acetic acid, propionic acid, hydroxyacetic acid, lactic acid, pyruvic acid, oxalic acid (ie ethanedioic acid), malonic acid, succinic acid (ie butanedioic acid), maleic acid, fumaric acid, malic acid (ie hydroxyl-butanedioic acid), tartaric acid, citric acid, methanesulfonic acid, ethanesulfonic acid , benzenesulfonic acid, p-toluenesulfonic acid, cyclamic acid, salicylic acid, p-aminosalicylic acid, pamoic acid, or combination thereof.

### DETAILED DESCRIPTION

### Definitions

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include the plural referents unless the context clearly indicates otherwise. The terms “include,” “such as,” and the like are intended to convey inclusion without limitation, unless otherwise specifically indicated.

As used herein, the term “comprising” also specifically includes embodiments “consisting of” and “consisting essentially of” the recited elements, unless specifically indicated otherwise.

The term “about” indicates and encompasses an indicated value and a range above and below that value. In certain embodiments, the term “about” indicates the designated value ±10%, ±5%, or ±1%. In certain embodiments, where applicable, the term “about” indicates the designated value(s)±one standard deviation of that value(s).

The term “intermediate” or “intermediate compound” refers to a compound which is produced in the course of a chemical synthesis, which is not itself the final product, but is used in further reactions which produce the final product. There may be many different intermediate compounds between the starting material and end product in the course of a complex synthesis.

The term “solvent” refers to a liquid material which may dissolve or suspend another material e.g. a reactant which may be a solid, gas, or liquid and provides a medium in which the reaction can occur. A solvent can be mixtures of different liquid materials.

The term “oxidoreductase” refers to an enzyme that catalyzes the transfer of electrons from one molecule, the reductant, also called the electron donor, to another, the oxidant, also called the electron acceptor. This group of enzymes usually utilizes NADP or NAD+ as cofactors. These enzymes fall into six categories: oxygenases, reductases, peroxidases, oxidases, hydroxylases, and dehydrogenases.

The term “dehydrogenase” or “DHase” refers to an enzyme belonging to the group of oxidoreductases that oxidizes a substrate by reducing an electron acceptor, usually NAD+/NADP+ or a flavin coenzyme such as FAD or FMN. They also catalyze the reverse reaction, for instance alcohol dehydrogenase not only oxidizes ethanol to acetaldehyde in animals but also produces ethanol from acetaldehyde in yeast.

The term “alcohol dehydrogenase” also referred as ketoreductases (KREDs).

The expression “pharmaceutically acceptable” refers to the ingredients of a pharmaceutical composition are compatible with each other and not deleterious to the subject to which it is administered.

The expression “pharmaceutically acceptable carrier, diluent, excipient and/or adjuvant” refers to a substance that does not produce an adverse, allergic or other untoward reaction when administered to an animal, preferably a human. It includes any and all inactive substances such as for example solvents, cosolvents, antioxidants, surfactants, stabilizing agents, emulsifying agents, buffering agents, pH modifying agents, preserving agents (or preservating agents), antibacterial and antifungal agents, isotonifiers, granulating agents or binders, lubricants, disintegrants, glidants, diluents or fillers, adsorbents, dispersing agents, suspending agents, coating agents, bulking agents, release agents, absorption delaying agents, sweetening agents, flavoring agents and the like. For human administration, preparations should meet sterility, pyrogenicity, general safety and purity standards as required by regulatory offices, such as, e.g., FDA Office or EMA.

The term “predrug”, as used herein, means any compound that will be modified to form a drug species, wherein the modification may take place either inside or outside of the body, and either before or after the predrug reaches the area of the body where administration of the drug is indicated.

The term “prodrug” as used herein means the pharmacologically acceptable derivatives of compounds of Formula (I), such as for example esters or amides, whose in vivo biotransformation product generates the biologically active drug. Prodrugs are generally characterized by increased bio-availability and are readily metabolized into biologically active compounds in vivo.

The term “sulfoxide” as used herein represents a group of formula “—S(O)—” wherein a sulfur atom is covalently attached to two carbon atoms.

The term “enantioselective oxidation conditions” means conditions that allow one enantiomer or set of diastereomers to be favored as an oxidation product for another enantiomer or set of diastereomers.

The term “chiral” means that the molecule cannot overlap on its mirror. The chiral center of the molecule is tetrahedral and has a non-conductive geometry, with each vertex of the tetrahedron being an atom different from the other vertices. Examples of chiral centers include carbon atoms to which four different substituents are attached. Another example of a chiral center is a sulfur atom in the sulfoxide moiety bound to sulfur, oxygen and two other different substituents.

The term “enantiomerically rich” means that one enantiomer or series of diastereomers is superior to the complementary enantiomer or series of diastereomers. Typically, whether a compound is enantiomerically enriched, for example, it integrates the area of two enantiomeric peaks, sums the areas, divides each area of each enantiomeric peak by the combined area of the two peaks, The dividend is expressed as a percentage of the total mixture of the two enantiomers and is determined by separating the mixture of the first enantiomer and the second enantiomer. If the first enantiomer is superior to the second enantiomer, the difference obtained by subtracting the percentage of the second enantiomer from the percentage of the first enantiomer is obtained as the percent enantiomeric abundance (% ee) of the first enantiomer. Display. The enantiomeric abundance may be about 1 to about 100% ee, preferably about 10 to about 100% ee, more preferably about 20 to about 100% ee, even more preferably about 50 to about 100% ee. have.

As used herein, the terms “biocatalysis,” “biocatalytic,” “biotransformation,” and “biosynthesis” refer to the use of enzymes to perform chemical reactions on organic compounds.

As used herein, “wild-type” and “naturally-occurring” refer to the form found in nature. For example a wild-type polypeptide or polynucleotide sequence is a sequence present in an organism that can be isolated from a source in nature and which has not been intentionally modified by human manipulation.

As used herein, “recombinant,” “engineered,” “non-naturally occurring,” and “variant,” when used with reference to a cell, nucleic acid, or polypeptide, refers to a material, or a material corresponding to the natural or native form of the material, that has been modified in a manner that would not otherwise exist in nature. In some embodiments, the cell, nucleic acid or polypeptide is identical a naturally occurring cell, nucleic acid or polypeptide, but is produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

The terms “cell-free biosynthesis system” and “CFB system” are used interchangeably and refer to the experimental design, set-up, apparatus, equipment, and materials, including a cell-free biosynthesis reaction mixture and cell extracts, as defined below, that carries out a cell-free biosynthesis reaction and produce a desired product, such as enantiomerically rich thiocarbamate derivatives.

### Thiocarbamate Derivatives as A2AR Inhibitors

The present disclosure relates to manufacturing process of thiocarbamate derivatives as A2A adenosine receptor (A2AR) inhibitors. Particularly, the present disclosure provides a method for production of high value key intermediates for A2AR inhibitors using cell-free biosynthesis system. The A2AR inhibitor is a thiocarbamate derivative, especially a thiocarbamate derivative as those disclosed in PCT/EP2018/058301, which is incorporated herein by reference.

The process according to the present disclosure allows to obtain a range of thiocarbamate derivatives, particular those including at least one sulfoxide.

The process according to the present disclosure is particularly advantageous for preparing enantiomerically rich thiocarbamate derivatives, preferably with a chiral center on the sulfur atom.

Particularly, the present disclosure provides a method for synthesis of enantiomerically rich key intermediates for thiocarbamate derivatives in a cell-free biosynthesis system having an enantiomeric excess of at least about 80%, at least 90%, at least 95%, at least 99%, or at least 99.9%.

The present disclosure also relates to enantiomeric enrichment process comprising overoxidation of an undesired enantiomer, whereby undesired enantiomer is converted to a sulfone. In some embodiments, the sulfone is removed by recrystallization.

In particular, by the process of the present disclosure synthetic routes for synthesis of key intermediates for thiocarbamate derivatives are made possible without requiring protective groups and without requiring the use of exotic reagents. Moreover, if desired, stereogenic centers can be efficiently created having an enantiomeric excess of at least about 80%,at least 90%, at least 95%, at least 99%, or at least 99.9%, thereby enhancing industrial acceptance and suitability of the process of the present disclosure.

Preferably the A2AR inhibitor that can be prepared by the processes described herein is a thiocarbamate derivative of formula (I) comprising at least one sulfoxide as described below.

In one embodiment, the thiocarbamate derivative A2AR inhibitor is of Formula (I):

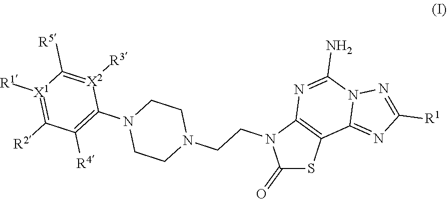

or a pharmaceutically acceptable salt or solvate thereof, wherein:

R¹ represents 5- or 6-membered heteroaryl or 5- or 6-membered aryl, wherein heteroaryl or aryl groups are optionally substituted by one or more substituent selected from C1-C6 alkyl (preferably methyl) and halo (preferably fluoro or chloro); preferably R¹ represents 5-membered heteroaryl; more preferably R¹ represents furyl;

X¹ and X² represent each independently C or N;

R^{1′} is absent when X¹ is N; or when X¹ is C, R^{1′} represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino or alkylsulfonealkyl; said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;

R^{2′} represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino, or alkylsulfonealkyl; said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;

or R^{1′} and R^{2′} form together with the atoms to which they are attached a 5- or 6-membered aryl ring, a 5- or 6-membered heteroaryl ring, a 5- or 6-membered cycloalkyl ring or a 5- or 6-membered heterocyclyl ring; optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;

wherein each of R^{1′} and R^{2′} comprises at least one sulfoxide as permitted by valency;

R^{3′} is absent when X² is N; or when X² is C, R^{3′} represents H or halo, preferably H or F;

R^{4′} represents H or halo, preferably H or F; and

R^{5′} represents H or halo, preferably H or F.

In one specific embodiment of the disclosure, R¹ represents 5- or 6-membered heteroaryl or 5- or 6-membered aryl, wherein heteroaryl or aryl groups are optionally substituted by one or more substituent selected from C1-C6 alkyl (preferably methyl) and halo (preferably fluoro or chloro). In a preferred embodiment, R¹ represents 5-membered heteroaryl; more preferably, R¹ represents furyl.

In one specific embodiment of the disclosure, X¹ and X² represent each independently C or N. In another specific embodiment, X¹ and X² both represent C.

In one specific embodiment of the disclosure, R^{1′} is absent when X¹ is N and R^{2′} comprises at least one sulfoxide.

In another specific embodiment, when X¹ is C, R^{1′} represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino or alkylsulfonealkyl; said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In a preferred embodiment, R^{1′} substituents are optionally substituted by one or more substituent selected from halo, hydroxy, alkyl, heterocyclylalkyl, hydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkoxy, amino, dialkylamino, aminoalkylcarbonylamino, aminocarbonylalkylamino, heterocyclylalkylaminocarbonyl, (aminocarbonylalkyl)(alkyl)amino, hydroxycarbonyl, aminocarbonyl, aminoalkylaminocarbonyl, alkylaminoalkylaminocarbonyl, dialkylaminoalkylaminocarbonyl, (alkylaminoalkyl)(alkyl)aminocarbonyl, heterocyclylcarbonyl, alkylsulfoxide and alkylsulfonealkyl.

In one specific embodiment of the disclosure, R^{2′} represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino, or alkylsulfonealkyl; said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In a preferred embodiment, R^{2′} substituents are optionally substituted by one or more substituent selected from oxo, halo, hydroxy, cyano, alkyl, heterocyclylalkyl, dihydroxyalkyl, dialkylaminoalkyl, heteroaryl, alkylheteroaryl, hydroxycarbonyl, alkyloxycarbonyl, aminocarbonyl, heterocyclylalkylaminocarbonyl, alkylaminoalkylcarbonyl, dialkylaminoalkylcarbonyl, alkylsulfoxide, alkylsulfonealkyl.

In a preferred embodiment, R^{1′} comprises at least one sulfoxide. In a more preferred embodiment, comprises only one sulfoxide, wherein sulfur atom is a chiral sulfur atom.

In another preferred embodiment, R^{2′} comprises at least one sulfoxide. More preferably, R^{2′} comprises only one sulfoxide, wherein sulfur atom is a chiral sulfur atom.

In another specific embodiment of the disclosure, R^{1′} and R^{2′} form together with the atoms to which they are attached a 5- or 6-membered aryl ring, a 5- or 6-membered heteroaryl ring, a 5- or 6-membered cycloalkyl ring or a 5- or 6-membered heterocyclyl ring; optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In one specific embodiment of the disclosure, R^{3′} is absent when X² is N. In another specific embodiment of the disclosure, when X² is C, R^{3′} represents H or halo. In a preferred embodiment, when X² is C, R^{3′} represents H or F.

In one specific embodiment of the disclosure, R^{4′} represents H or halo. In a preferred embodiment, R^{4′} represents H or F.

In one specific embodiment of the disclosure, R^{5′} represents H or halo. In a preferred embodiment, R^{5′} represents H or F.

In one embodiment, preferred compounds of Formula (I) are those of Formula (Ia):

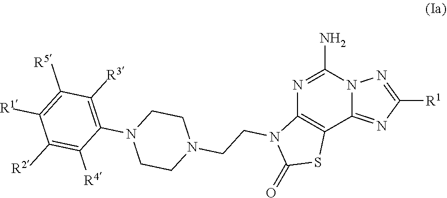

or a pharmaceutically acceptable salt or solvate thereof, wherein R¹, R^{1′}, R^{2′}, R^{3′}, R^{4′} and R^{5′} are as defined in Formula (I).

In one embodiment, preferred compounds of Formula (Ia) are those of Formula (Ia-1):

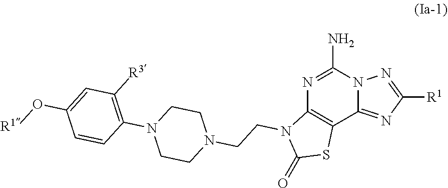

or a pharmaceutically acceptable salt or solvate thereof, wherein:

R¹ and R^{3′} are as defined in Formula (I); and

R^{1″} represents an alkyl or heterocyclyl group substituted by one or more group selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl, wherein R^{1″} comprises at least one sulfoxide.

In a preferred embodiment, R^{1″} represents an alkyl or heterocyclyl group substituted by one or more group selected from alkylsulfoxide, alkylsulfoxidealkyl, alkylsulfonyl and alkylsulfonealkyl.

In one embodiment, preferred compounds of Formula (Ia-1) are those of Formula (Ia-1a):

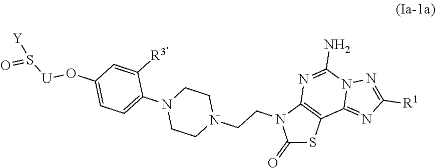

or a pharmaceutically acceptable salt or solvate thereof, wherein:

R¹ and R^{3′} are as defined in Formula (Ia);

U represents alkylene, arylene, heteroarylene or heterocyclyene optionally substituted by one or more substituent selected from halo, hydroxy, alkyl, heterocyclylalkyl, hydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkoxy, amino, dialkylamino, aminoalkylcarbonylamino, aminocarbonylalkylamino, heterocyclylalkylaminocarbonyl, (aminocarbonylalkyl)(alkyl)amino, hydroxycarbonyl, aminocarbonyl, aminoalkylaminocarbonyl, alkylaminoalkylaminocarbonyl, dialkylaminoalkylaminocarbonyl, (alkylaminoalkyl)(alkyl)aminocarbonyl, heterocyclylcarbonyl, alkylsulfoxide and alkylsulfonealkyl;

Y represents represents an alkyl, arylene, heteroarylene or heterocyclyene optionally substituted by one or more group selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;

or U and Y form together with the atoms to which they are attached an aryl ring, a heteroaryl ring, a cycloalkyl ring or a heterocyclyl ring; optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In one embodiment, preferred compounds of Formula (Ia-1) are those of Formula (Ia-1b):

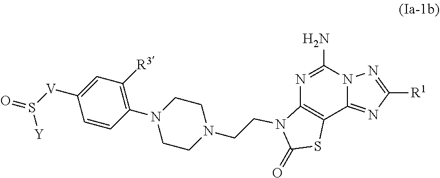

or a pharmaceutically acceptable salt or solvate thereof, wherein:

R¹ and R^{3′} are as defined in Formula (Ia) and Y is as defined as in Formula (Ia-1a); and

V represents a bond, alkylene, arylene, heteroarylene or heterocyclyene optionally substituted by one or more substituent selected from halo, hydroxy, alkyl, heterocyclylalkyl, hydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkoxy, amino, dialkylamino, aminoalkylcarbonylamino, aminocarbonylalkylamino, heterocyclylalkylaminocarbonyl, (aminocarbonylalkyl)(alkyl)amino, hydroxycarbonyl, aminocarbonyl, aminoalkylaminocarbonyl, alkylaminoalkylaminocarbonyl, dialkylaminoalkylaminocarbonyl, (alkylaminoalkyl)(alkyl)aminocarbonyl, heterocyclylcarbonyl, alkylsulfoxide and alkylsulfonealkyl;

or V and Y form together with the atoms to which they are attached an aryl ring, a heteroaryl ring, a cycloalkyl ring or a heterocyclyl ring; optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In one embodiment, preferred compounds of Formula (Ia) are those of Formula (Ia-2):

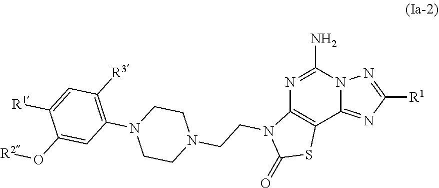

or a pharmaceutically acceptable salt or solvate thereof, wherein:

R¹ and R^{3′} are as defined in Formula (Ia);

R^{1′} represents H or halo, preferably H or F; and

R^{2″} represents an alkyl or heterocyclyl group substituted by one or more group selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl, wherein R^{2″} comprises at least one sulfoxide.

In one specific embodiment of the disclosure, R^{1′} represents H or halo. In a preferred embodiment, R^{1′} represents H or F.

In one specific embodiment of the disclosure, R^{3′} represents H or halo. In a preferred embodiment, R^{1′} represents H or F.

In a preferred embodiment, R¹′ and R^{3′} represents F.

In one specific embodiment of the disclosure, R^{2″} represents an alkyl or heterocyclyl group substituted by one or more group selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In a preferred embodiment, R^{2″} represents an alkyl or heterocyclyl group substituted by one or more group selected from hydroxy, cyano, heteroaryl, alkylheteroaryl, alkyne, hydroxycarbonyl, alkyloxycarbonyl, aminocarbonyl, alkylaminoalkylcarbonyl, dialkylaminoalkylcarbonyl, alkylsulfoxide, alkylsulfonealkyl.

In one embodiment, preferred compounds of Formula (Ia-2) are those of Formula (Ia-2a):

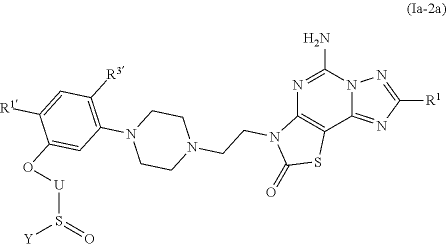

or a pharmaceutically acceptable salt or solvate thereof, wherein R¹, R^{1′}, R^{3′}, are as defined in Formula (I) and U, Y are defined as in Formula (Ia-1a).

In a preferred embodiment, R^{1′} and R^{3′} represents halo.

In one embodiment, preferred compounds of Formula (Ia-1) are those of Formula (Ia-1c) or (Ia-1d):

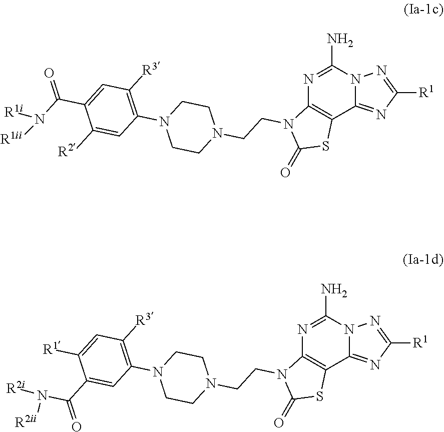

or a pharmaceutically acceptable salt or solvate thereof, wherein:

R¹ and R^{3′} are as defined in Formula (Ia);

R^{1′} represents H or halo, preferably H or F;

R^{2′} represents H or halo, preferably H or F;

R¹ⁱ or R¹ⁱⁱ comprises at least one sulfoxide;

R¹ⁱ and R¹ⁱⁱ represent each independently hydrogen, hydroxy, alkyl, alkenyl, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynealkyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxidealkyl or alkylsulfonealkyl, wherein R¹ⁱ or R¹ⁱⁱ comprises at least one sulfoxide; and

R²ⁱ and R²ⁱⁱ represent each independently hydrogen, hydroxy, alkyl, alkenyl, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynealkyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxidealkyl or alkylsulfonealkyl, wherein R²ⁱ or R²ⁱⁱ comprises at least one sulfoxide.

In one specific embodiment of the disclosure, R^{1′} represents H or halo. In a preferred embodiment, R^{1′} represents H or F.

In one specific embodiment of the disclosure, R^{2′} represents H or halo. In a preferred embodiment, R^{2′} represents H or F.

In one specific embodiment of the disclosure, R¹ⁱ and R¹ⁱⁱ represent each independently hydrogen, hydroxy, alkyl, alkenyl, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynealkyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxidealkyl or alkylsulfonealkyl.

In a preferred embodiment, R¹ⁱ and R¹ⁱⁱ represent each independently hydrogen, alkyl, heterocyclylalkyl, hydroxyalkyl, hydroxyalkylaminoalkyl, aminoalkyl, alkylaminoalkyl, dialkylaminoalkyl, (heterocyclyl)(alkyl)aminoalkyl or heterocyclylalkylaminocarbonyl.

In one specific embodiment of the disclosure, R²ⁱ and R²ⁱⁱ represent each independently hydrogen, hydroxy, alkyl, alkenyl, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynealkyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxidealkyl or alkylsulfonealkyl.

In a preferred embodiment, R²ⁱ and R²ⁱⁱ represent each independently hydrogen, alkyl, heterocyclylalkyl, dihydroxyalkyl, dialkylaminoalkyl or heterocyclylalkylaminocarbonyl. In a preferred embodiment, R²ⁱ and R²ⁱⁱ represent each independently hydrogen, alkyl or dialkylaminoalkyl.

Particularly preferred compounds of Formula (I) of the disclosure are those listed in Table 1 hereafter.

TABLE 1

| Cpd | | | |
|---|---|---|---|
| no | Structure | Chemical name | MW |
| 1 | 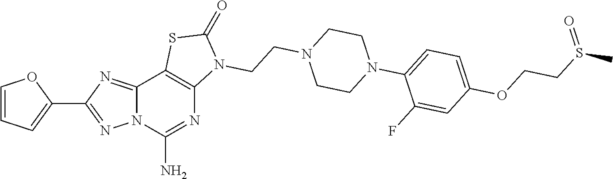 [SMILES: C[S@](=O)CCOc1ccc(N2CCN(CCn3c(=O)sc4c3nc(N)n3nc(-c5ccco5)nc43)CC2)c(F)c1] | (S)-5-amino-3- (2-(4-(2-fluoro- 4-(2-(methylsulfinyl) ethoxy)phenyl) piperazin-1- yl)ethyl)-8-(furan-2- yl)thiazolo[5,4- e][1,2,4]triazolo[1,5- c]pyrimidin-2(3H)-one | 586.66 |
| 2 | 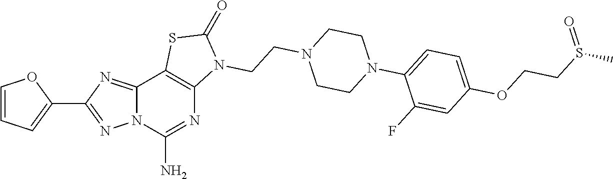 [SMILES: C[S@@](=O)CCOc1ccc(N2CCN(CCn3c(=O)sc4c3nc(N)n3nc(-c5ccco5)nc43)CC2)c(F)c1] | (R)-5-amino-3- (2-(4-(2-fluoro- 4-(2-(methylsulfinyl) ethoxy)phenyl)- piperazin-1- yl)ethyl)-8-(furan-2- yl)thiazolo[5,4- e][1,2,4]triazolo[1,5- c]pyrimidin-2(3H)-one | 586.66 |
| 3a | 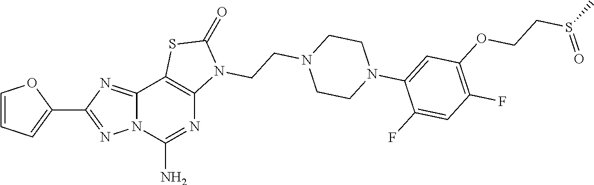 [SMILES: C[S@](=O)CCOc1cc(N2CCN(CCn3c(=O)sc4c3nc(N)n3nc(-c5ccco5)nc43)CC2)c(F)cc1F] | (+)-5-amino-3- (2-(4-(2,4- difluoro-5-(2- (methylsulfinyl) ethoxy)phenyl) piperazin-1- yl)ethyl)-8-(furan-2- yl)thiazolo[5,4- e][1,2,4]triazolo[1,5- c]pyrimidin-2(3H)-one | 604.65 |
| 3b | 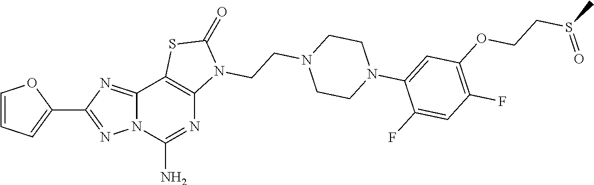 [SMILES: C[S@@](=O)CCOc1cc(N2CCN(CCn3c(=O)sc4c3nc(N)n3nc(-c5ccco5)nc43)CC2)c(F)cc1F] | (−)-(R)-5-amino- 3-(2-(4-(2,4- difluoro-5-(2- (methylsulfinyl) ethoxy)phenyl) piperazin-1- yl)ethyl)-8-(furan-2- yl)thiazolo[5,4- e][1,2,4]triazolo[1,5- c]pyrimidin-2(3H)-one | 604.65 |
| 4 | 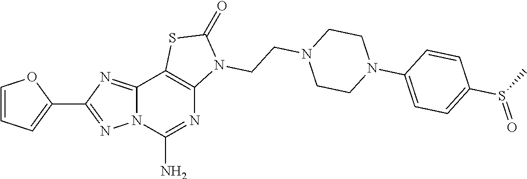 [SMILES: C[S@](=O)c1ccc(N2CCN(CCn3c(=O)sc4c3nc(N)n3nc(-c5ccco5)nc43)CC2)cc1] | (S)-5-amino-8- (furan-2-yl)-3- (2-(4-(4- (methylsulfinyl) phenyl)piperazin-1- yl)ethyl)thiazolo[5,4- e][1,2,4]triazolo[1,5- c]pyrimidin-2(3H)-one | 524.62 |
| 5 | 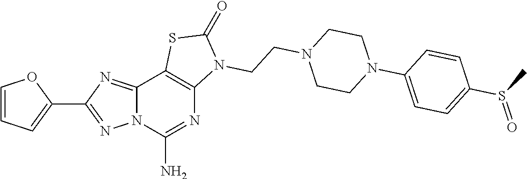 [SMILES: C[S@@](=O)c1ccc(N2CCN(CCn3c(=O)sc4c3nc(N)n3nc(-c5ccco5)nc43)CC2)cc1] | (R)-5-amino-8- (furan-2-yl)-3- (2-(4-(4- (methylsulfinyl) phenyl)piperazin-1- yl)ethyl)thiazolo[5,4- e][1,2,4]triazolo[1,5- c]pyrimidin-2(3H)-one | 524.62 |
| 6 | 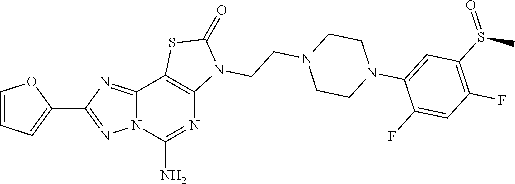 [SMILES: C[S@](=O)c1cc(N2CCN(CCn3c(=O)sc4c3nc(N)n3nc(-c5ccco5)nc43)CC2)c(F)cc1F] | (S)-5-amino-3- (2-(4-(2,4- difluoro-5- (methylsulfinyl) phenyl)piperazin- 1-yl)ethyl)-8-(furan-2- yl)thiazolo[5,4- e][1,2,4]triazolo[1,5- c]pyrimidin-2(3H)-one | 560.60 |
| 7 | 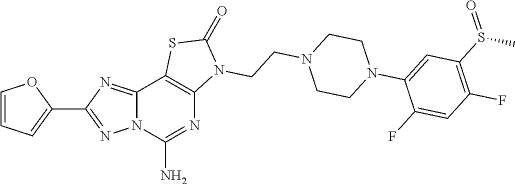 [SMILES: C[S@@](=O)c1cc(N2CCN(CCn3c(=O)sc4c3nc(N)n3nc(-c5ccco5)nc43)CC2)c(F)cc1F] | (R)-5-amino-3- (2-(4-(2,4- difluoro-5- (methylsulfinyl) phenyl)piperazin- 1-yl)ethyl)-8-(furan-2- yl)thiazolo[5,4- e][1,2,4]triazolo[1,5- c]pyrimidin-2(3H)-one | 560.60 |
| 8 | 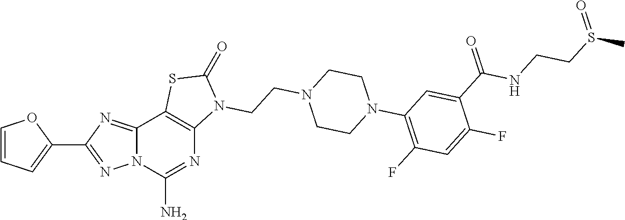 [SMILES: C[S@](=O)CCNC(=O)c1cc(N2CCN(CCn3c(=O)sc4c3nc(N)n3nc(-c5ccco5)nc43)CC2)c(F)cc1F] | (S)-5-(4-(2-(5- amino-8-(furan- 2-yl)-2- oxothiazolo[5,4- e][1,2,4]triazolo[1,5- c]pyrimidin-3(2H)- yl)ethyl)piperazin- 1-yl)-2,4- difluoro-N-(2- (methylsulfinyl)ethyl) benzamide | 631.68 |
| 9 | 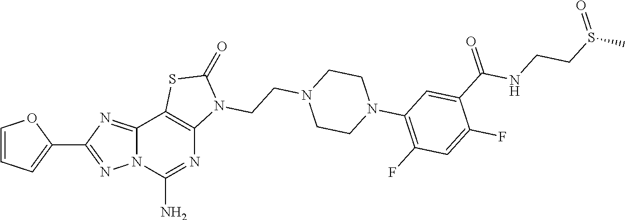 [SMILES: C[S@@](=O)CCNC(=O)c1cc(N2CCN(CCn3c(=O)sc4c3nc(N)n3nc(-c5ccco5)nc43)CC2)c(F)cc1F] | (R)-5-(4-(2-(5- amino-8-(furan- 2-yl)-2- oxothiazolo[5,4- e][1,2,4]triazolo[1,5- c]pyrimidin-3(2H)- yl)ethyl)piperazin- 1-yl)-2,4- difluoro-N-(2- (methylsulfinyl)ethyl) benzamide | 631.68 |
| 10 | 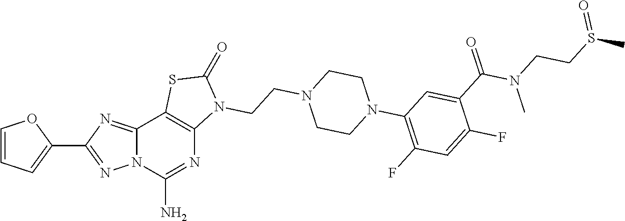 [SMILES: CN(CC[S@](C)=O)C(=O)c1cc(N2CCN(CCn3c(=O)sc4c3nc(N)n3nc(-c5ccco5)nc43)CC2)c(F)cc1F] | (S)-5-(4-(2-(5- amino-8-(furan- 2-yl)-2- oxothiazolo[5,4- e][1,2,4]triazolo[1,5- c]pyrimidin-3(2H)- yl)ethyl)piperazin- 1-yl)-2,4-difluoro- N-methyl-N-(2- (methylsulfinyl)ethyl) benzamide | 645.70 |
| 11 | 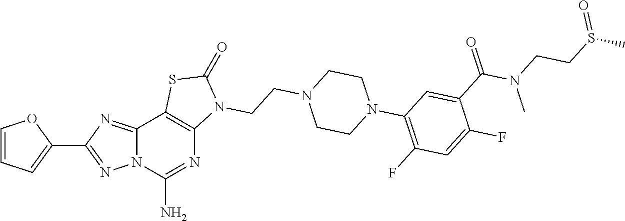 [SMILES: CN(CC[S@@](C)=O)C(=O)c1cc(N2CCN(CCn3c(=O)sc4c3nc(N)n3nc(-c5ccco5)nc43)CC2)c(F)cc1F] | (R)-5-(4-(2-(5- amino-8-(furan- 2-yl)-2- oxothiazolo[5,4- e][1,2,4]triazolo[1,5- c]pyrimidin-3(2H)- yl)ethyl)piperazin- 1-yl)-2,4-difluoro- N-methyl-N-(2- (methylsulfinyl)ethyl) benzamide | 645.70 |
| 12 | 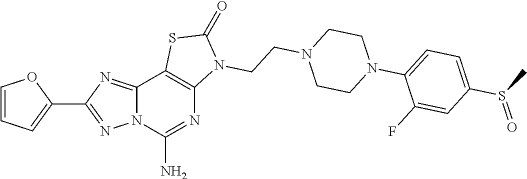 [SMILES: C[S@@](=O)c1ccc(N2CCN(CCn3c(=O)sc4c3nc(N)n3nc(-c5ccco5)nc43)CC2)c(F)c1] | (R)-5-amino-3- (2-(4-(2-fluoro- 4-(methylsulfinyl) phenyl)piperazin- 1-yl)ethyl)-8-(furan-2- yl)thiazolo[5,4- e][1,2,4]triazolo[1,5- c]pyrimidin-2(3H)-one | 542.61 |
| 13 | 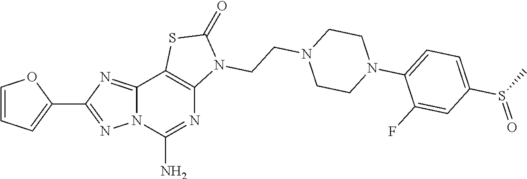 [SMILES: C[S@](=O)c1ccc(N2CCN(CCn3c(=O)sc4c3nc(N)n3nc(-c5ccco5)nc43)CC2)c(F)c1] | (S)-5-amino-3- (2-(4-(2-fluoro- 4-(methylsulfinyl) phenyl)piperazin- 1-yl)ethyl)-8-(furan-2- yl)thiazolo[5,4- e][1,2,4]triazolo[1,5- c]pyrimidin-2(3H)-one | 542.61 |
| 14 | 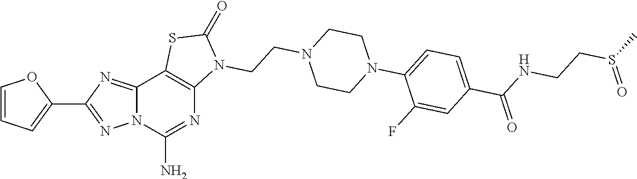 [SMILES: C[S@](=O)CCNC(=O)c1ccc(N2CCN(CCn3c(=O)sc4c3nc(N)n3nc(-c5ccco5)nc43)CC2)c(F)c1] | (S)-4-(4-(2-(5- amino-8-(furan- 2-yl)-2- oxothiazolo[5,4- e][1,2,4]triazolo[1,5- c]pyrimidin-3(2H)- yl)ethyl)piperazin- 1-yl)-3- fluoro-N-(2- (methylsulfinyl)ethyl) benzamide | 613.69 |
| 15 | 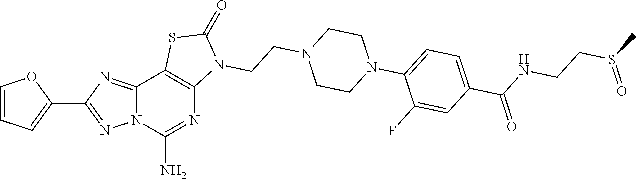 [SMILES: C[S@@](=O)CCNC(=O)c1ccc(N2CCN(CCn3c(=O)sc4c3nc(N)n3nc(-c5ccco5)nc43)CC2)c(F)c1] | (R)-4-(4-(2-(5- amino-8-(furan- 2-yl)-2- oxothiazolo[5,4- e][1,2,4]triazolo[1,5- c]pyrimidin-3(2H)- yl)ethyl)piperazin- 1-yl)-3- fluoro-N-(2- (methylsulfinyl)ethyl) benzamide | 613.69 |
| 16 | 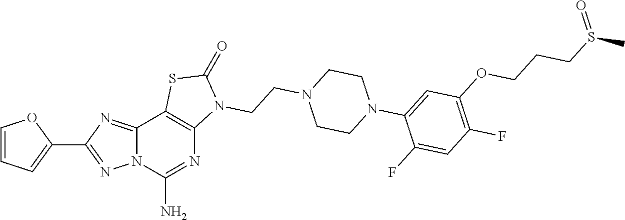 [SMILES: C[S@](=O)CCCOc1cc(N2CCN(CCn3c(=O)sc4c3nc(N)n3nc(-c5ccco5)nc43)CC2)c(F)cc1F] | (S)-5-amino-3- (2-(4-(2,4- difluoro-5-(3- (methylsulfinyl) propoxy)phenyl) piperazin-1- yl)ethyl)-8-(furan-2- yl)thiazolo[5,4- e][1,2,4]triazolo[1,5- c]pyrimidin-2(3H)-one | 618.68 |

and pharmaceutically acceptable salts and solvates thereof.

In Table 1, the term “Cpd” means compound.

The compounds of Table 1 were named using ChemBioDraw® Ultra version 12.0 (PerkinElmer).

In one embodiment, a compound of Formula (I) is Compound 3a. In some embodiments, Compound 3a is (+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one. In some embodiments, the compound denoted as “Compound 3a” may be referred to as (+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one, (+)-(S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one, or (S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one interchangely. It will be readily understood by a person of ordinary skill in that art that (+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one refers to an enantiomer of 5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one. The present disclosure has drawn (+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one in the (S) configuration. The present disclosure additionally contemplates (R)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one.

The present disclosure includes Intermediate B. In some embodiments, the compound referred to as Intermediate B is (+)-1-(2,4-difluoro-5-(2-(methylsulfinyl)ethoxy)phenyl)piperazine. In some embodiments, the compound denoted as “Intermediate B” may also be referred to as (+)-1-(2,4-difluoro-5-(2-(methylsulfinyl)ethoxy)phenyl)piperazine, (+)-(S)-1-(2,4-difluoro-5-(2-(methylsulfinyl)ethoxy)phenyl)piperazine, or (S)-1-(2,4-difluoro-5-(2-(methylsulfinyl)ethoxy)phenyl)piperazine interchangely. It will be readily understood by a person of ordinary skill in that art that (+)-1-(2,4-difluoro-5-(2-(methylsulfinyl)ethoxy)phenyl)piperazine refers to an enantiomer of 1-(2,4-difluoro-5-(2-(methylsulfinyl)ethoxy)phenyl)piperazine. The present disclosure has drawn (+)-1-(2,4-difluoro-5-(2-(methylsulfinyl)ethoxy)phenyl)piperazine in the (S) configuration. The present disclosure additionally contemplates (R)-1-(2,4-difluoro-5-(2-(methylsulfinyl)ethoxy)phenyl)piperazine.

In one embodiment, the compound of Formula (I) is selected from (S)-5-amino-3-(2-(4-(2-fluoro-4-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2-fluoro-4-(2-(methylsulfinyl)ethoxy)phenyl)-piperazin-1-yl)ethyl)-8-(furan-2-yl)thiazolo[5,4-e][1,2,4]triazolo[1,5-c]pyrimidin-2(3H)-one;
(−)-(R)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(S)-5-amino-8-(furan-2-yl)-3-(2-(4-(4-(methylsulfinyl)phenyl)piperazin-1-yl)ethyl)thiazolo[5,4-e][1,2,4]triazolo[1,5-c]pyrimidin-2(3H)-one;
(R)-5-amino-8-(furan-2-yl)-3-(2-(4-(4-(methylsulfinyl)phenyl)piperazin-1-yl)ethyl)thiazolo[5,4-e][1,2,4]triazolo[1,5-c]pyrimidin-2(3H)-one;
(S)-5-amino-3-(2-(4-(2,4-difluoro-5-(methylsulfinyl)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2,4-difluoro-5-(methylsulfinyl)phenyl)piperazin-1-yl)ethyl)-8-(furan-2-yl)thiazolo[5,4-e][1,2,4]triazolo[1,5-c]pyrimidin-2(3H)-one;
(S)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-(2-(methylsulfinyl)ethyl)benzamide;
(R)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-(2-(methylsulfinyl)ethyl)benzamide;
(S)-5-(4-(2-(5-amino-8-(furan-2-yl)-2-oxothiazolo[5,4-e][1,2,4]triazolo[1,5-c]pyrimidin-3(2H)-yl)ethyl)piperazin-l-yl)-2,4-difluoro-N-methyl-N-(2-(methylsulfinyl)ethyl)benzamide;
(R)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-methyl-N-(2-(methylsulfinyl)ethyl)benzamide;
(R)-5-amino-3-(2-(4-(2-fluoro-4-(methylsulfinyl)phenyl)piperazin-1-yl)ethyl)-8-(furan-2-yl)thiazolo[5,4-e][1,2,4]triazolo[1,5-c]pyrimidin-2(3H)-one;
(S)-5-amino-3-(2-(4-(2-fluoro-4-(methylsulfinyl)phenyl)piperazin-1-yl)ethyl)-8-(furan-2-yl)thiazolo[5,4-e][1,2,4]triazolo[1,5-c]pyrimidin-2(3H)-one;
(S)-4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methylsulfinyl)ethyl)benzamide;
(R)-4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methylsulfinyl)ethyl)benzamide;
(S)-5-amino-3-(2-(4-(2,4-difluoro-5-(3-(methylsulfinyl)propoxy)phenyl)piperazin-1-yl)ethyl)-8-(furan-2-yl)thiazolo[5,4-e][1,2,4]triazolo[1,5-c]pyrimidin-2(3H)-one;

In one embodiment, the present disclosure also relates to manufacturing of diastereomers, enantiomers, salts, solvates, polymorphs, multi-component complexes and liquid crystals of compounds of Formula (I) and subformula thereof.

In a preferred embodiment, the manufacturing method mentioned in this disclosure yields to enantiomerically rich compounds of Formula (I) with a chiral center on the sulfur atom.

In one embodiment, the present disclosure also relates to manufacturing of polymorphs and crystal habits of compounds of Formula (I) and subformula thereof, prodrugs and isomers thereof (including optical, geometric and tautomeric isomers) and isotopically-labeled compounds of Formula (I) and subformula thereof.

In one embodiment, the present disclosure also relates to manufacturing of the compounds of Formula (I) and subformula thereof may contain an asymmetric center (a chiral center) and thus may exist as different stereoisomeric forms. Accordingly, the present disclosure includes all possible stereoisomers and includes not only racemic compounds but the individual enantiomers and their non-racemic mixtures as well. When a compound is desired as a single enantiomer, such may be obtained by stereospecific synthesis, by resolution of the final product or any convenient intermediate, or by chiral chromatographic methods as each are known in the art. Resolution of the final product, an intermediate, or a starting material may be performed by any suitable method known in the art.

The compounds of the disclosure may be in the form of pharmaceutically acceptable salts. Pharmaceutically acceptable salts of the compounds of Formula (I) and subformula thereof include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine, 2-(diethylamino)ethanol, ethanolamine, morpholine, 4-(2-hydroxyethyl)morpholine and zinc salts. Hemisalts of acids and bases may also be formed, for example, hemisulphate and hemicalcium salts. Preferred, pharmaceutically acceptable salts include hydrochloride/chloride, hydrobromide/bromide, bisulphate/sulphate, nitrate, citrate, tosylate, esylate and acetate. In a particularly preferred embodiment, the compounds of Formula (I) is under the form of a HCl salt or esylate salt.

When the compounds of the disclosure contain an acidic group as well as a basic group the compounds of the disclosure may also form internal salts, and such compounds are within the scope of the disclosure. When the compounds of the disclosure contain a hydrogen-donating heteroatom (e.g. NH), the disclosure also covers salts and/or isomers formed by transfer of said hydrogen atom to a basic group or atom within the molecule.

Pharmaceutically acceptable salts of compounds of Formula (I) and subformula thereof may be prepared by one or more of these methods:
(i) by reacting the compound of Formula (I) with the desired acid;
(ii) by reacting the compound of Formula (I) with the desired base;
(iii) by removing an acid- or base-labile protecting group from a suitable precursor of the compound of Formula (I) or by ring-opening a suitable cyclic precursor, for example, a lactone or lactam, using the desired acid; or
(iv) by converting one salt of the compound of Formula (I) to another by reaction with an appropriate acid or by means of a suitable ion exchange column.

All these reactions are typically carried out in solution. The salt, may precipitate from solution and be collected by filtration or may be recovered by evaporation of the solvent. The degree of ionization in the salt may vary from completely ionized to almost non-ionized.

The compounds of the present disclosure may be administered in the form of pharmaceutically acceptable salts. The term “pharmaceutically acceptable salt” is intended to include all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations. Depending on the particular functionality of the compound of the present disclosure, pharmaceutically acceptable salts of the compounds of this disclosure include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylene-diamine, chloroprocaine, diethanolamine, procaine, N-benzylphenethyl-amine, diethylamine, piperazine, tris(hydroxymethyl)aminomethane, and tetramethylammonium hydroxide.

These salts may be prepared by standard procedures, e.g. by reacting a free acid with a suitable organic or inorganic base. Where a basic group is present, such as amino, an acidic salt, i.e. hydrochloride, hydrobromide, acetate, palmoate, esylate, tosylate and the like, can be used as the dosage form.

In addition, although generally, with respect to the salts of the compounds of the disclosure, pharmaceutically acceptable salts are preferred, it should be noted that the disclosure in its broadest sense also included non-pharmaceutically acceptable salts, which may for example be used in the isolation and/or purification of the compounds of the disclosure. For example, salts formed with optically active acids or bases may be used to form diastereoisomeric salts that can facilitate the separation of optically active isomers of the compounds of Formula (I) above.

The compounds of the disclosure may be in the form of pharmaceutically acceptable solvates. Pharmaceutically acceptable solvates of the compounds of Formula (I) and subformula thereof contains stoichiometric or sub-stoichiometric amounts of one or more pharmaceutically acceptable solvent molecule such as ethanol or water. The term “hydrate” refers to when the said solvent is water.

The disclosure also generally covers all pharmaceutically acceptable predrugs and prodrugs of the compounds of Formula (I) and subformula thereof.

Also, in the case of an alcohol group being present, pharmaceutically acceptable esters can be employed, e.g. acetate, maleate, pivaloyloxymethyl, and the like, and those esters known in the art for modifying solubility or hydrolysis characteristics for use as sustained release or prodrug formulations.

### Pharmaceutical Composition

The disclosure also relates to pharmaceutical compositions comprising a compound of Formula I or a pharmaceutically acceptable salt and solvate thereof which is manufactured by a process comprising cell free enzymatic biotransformation and at least one pharmaceutically acceptable carrier, diluent, excipient and/or adjuvant. As indicated above, the disclosure also covers pharmaceutical compositions which contain, in addition to a compound of the present disclosure, a pharmaceutically acceptable salt and solvate thereof as active ingredient, additional therapeutic agents and/or active ingredients.

Another object of this disclosure is a medicament comprising at least one compound of the disclosure, or a pharmaceutically acceptable salt and solvate thereof, as active ingredient.

According to a further feature of the present disclosure there is provided the use of a compound of Formula I or a pharmaceutically acceptable salt and solvate thereof for the manufacture of a medicament for modulating A2A activity in a patient, in need of such treatment, which comprises administering to said patient an effective amount of compound of the present disclosure, or a pharmaceutically acceptable salt and solvate thereof.

Generally, for pharmaceutical use, the compounds of the disclosure may be formulated as a pharmaceutical preparation comprising at least one compound of the disclosure and at least one pharmaceutically acceptable carrier, diluent, excipient and/or adjuvant, and optionally one or more further pharmaceutically active compounds.

By means of non-limiting examples, such a formulation may be in a form suitable for oral administration, for parenteral administration (such as by intravenous, intramuscular or subcutaneous injection or intravenous infusion), for topical administration (including ocular), for administration by inhalation, by a skin patch, by an implant, by a suppository, etc. Such suitable administration forms—which may be solid, semi-solid or liquid, depending on the manner of administration—as well as methods and carriers, diluents and excipients for use in the preparation thereof, will be clear to the skilled person; reference is made to the latest edition of Remington's Pharmaceutical Sciences.

Some preferred, but non-limiting examples of such preparations include tablets, pills, powders, lozenges, sachets, cachets, elixirs, suspensions, emulsions, solutions, syrups, aerosols, ointments, cremes, lotions, soft and hard gelatin capsules, suppositories, drops, sterile injectable solutions and sterile packaged powders (which are usually reconstituted prior to use) for administration as a bolus and/or for continuous administration, which ma be formulated with carriers, excipients, and diluents that are suitable per se for such formulations, such as lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, (sterile) water, methylcellulose, methyl- and propylhydroxybenzoates, talc, magnesium stearate, edible oils, vegetable oils and mineral oils or suitable mixtures thereof. The formulations can optionally contain other substances that are commonly used in pharmaceutical formulations, such as lubricating agents, wetting agents, emulsifying and suspending agents, dispersing agents, desintegrants, bulking agents, fillers, preserving agents, sweetening agents, flavoring agents, flow regulators, release agents, etc.. The compositions may also be formulated so as to provide rapid, sustained or delayed release of the active compound(s) contained therein.

The pharmaceutical preparations of the disclosure are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use.

Depending on the condition to be prevented or treated and the route of administration, the active compound of the disclosure may be administered as a single daily dose, divided over one or more daily doses, or essentially continuously, e.g. using a drip infusion.

### Process for Manufacturing of A2A Inhibitors

The present disclosure relates to synthesis of enantiomerically rich key drug intermediates. More particularly, the present disclosure relates to the use of enzymatic biotransformation process in the manufacture of thiocarbamate derivatives which are useful as A2A adenosine receptor (A2AR) inhibitors.

The conventional small-scale synthesis (mg-scale) of key drug intermediates and A2A inhibitors mentioned in this disclosure is hereby incorporated in reference PCT/EP2018/058301. The relevant compounds disclosed in PCT/EP2018/058301 are the compounds comprising at least one sulfoxide. In a preferred embodiment, sulfur is chiral.

In general, the synthesis pathways for any individual compound of Formula (I) will depend on the specific substituents of each molecule and upon the ready availability of intermediates necessary; again such factors being appreciated by those of ordinary skill in the art.

According to a further general process, compounds of Formula Ia can be converted to alternative compounds of Formula Ia, employing suitable interconversion techniques well known by a person skilled in the art.

Compounds of the Formula Ia and related formulae can furthermore be obtained by liberating compounds of the Formula Ia from one of their functional derivatives by treatment with a solvolysing or hydrogenolysing agent. Preferred starting materials for the solvolysis or hydrogenolysis are those which conform to the Formula Ia and related formula, but contain corresponding protected amino and/or hydroxyl groups instead of one or more free amino and/or hydroxyl groups, preferably those which carry an amino-protecting group instead of an H atom bonded to an N atom, in particular those which carry an R*—N group, in which R* denotes an amino-protecting group, instead of an FiN group, and/or those which carry a hydroxyl-protecting group instead of the H atom of a hydroxyl group, for example those which conform to the Formula I, but carry a —COOR** group, in which R** denotes a hydroxyl-protecting group, instead of a —COOH group.

It is also possible for a plurality of—identical or different—protected amino and/or hydroxyl groups to be present in the molecule of the starting material. If the protecting groups present are different from one another, they can in many cases be cleaved off selectively.

The term “amino-protecting group” is known in general terms and relates to groups which are suitable for protecting (blocking) an amino group against chemical reactions, but which are easy to remove after the desired chemical reaction has been carried out elsewhere in the molecule. Typical of such groups are, in particular, unsubstituted or substituted acyl, aryl,aralkoxymethyl or aralkyl groups. Since the amino-protecting groups are removed after the desired reaction (or reaction sequence), their type and size are furthermore not crucial; however, preference is given to those having 1-20, in particular 1-8, carbon atoms. The term “acyl group” is to be understood in the broadest sense in connection with the present process. It includes acyl groups derived from aliphatic, araliphatic, aromatic or heterocyclic carboxylic acids or sulfonic acids, and, in particular, alkoxy-carbonyl, aryloxycarbonyl and especially aralkoxycarbonyl groups. Examples of such acyl groups are alkanoyl, such as acetyl, propionyl and butyryl; aralkanoyl, such as phenylacetyl; aroyl, such as benzoyl and tolyl; aryloxyalkanoyl, such as POA; alkoxycarbonyl, such as methoxy-′carbonyl, ethoxycarbonyl, 2,2,2-trichloroethoxycarbonyl, BOC (tert-butoxycarbonyl) and 2-iodoethoxycarbonyl aralkoxycarbonyl, such as CBZ (“carbobenzoxy”), 4-methoxybenzyloxycarbonyl and FMOC; and arylsulfonyl, such as Mtr. Preferred aminoprotecting groups are BOC and Mtr, further-more CBZ, Fmoc, benzyl and acetyl.

The term “hydroxyl-protecting group” is likewise known in general terms and relates to groups which are suitable for protecting a hydroxyl group against chemical reactions, but are easy to remove after the desired chemical reaction has been carried out elsewhere in the molecule. Typical of such groups are the above-mentioned unsubstituted or substituted aryl, aralkyl or acyl groups, furthermore also alkyl groups. The nature and size of the hydroxyl protecting groups are not crucial since they are removed again after the desired chemical reaction or reaction sequence; preference is given to groups having 1-20, in particular 1-10, carbon atoms. Examples of hydroxyl-protecting groups are, inter alia, benzyl, 4-methoxybenzyl, p-nitrobenzoyl, p-toluenesulfonyl, tert-butyl and acetyl, where benzyl and tert-butyl are particularly preferred.

The compounds of the Formula Ia and related formulae are liberated from their functional derivatives—depending on the protecting group used—for example strong inorganic acids, such as hydrochloric acid, perchloric acid or sulfuric acid, strong organic carboxylic acids, such as trichloroacetic acid, TFA or sulfonic acids, such as benzene- or p-toluenesulfonic acid. The presence of an additional inert solvent is possible, but is not always necessary.

Suitable inert solvents are preferably organic, for example carboxylic acids, such as acetic acid, ethers, such as tetrahydrofuran or dioxane, amides, such as DMF, halogenated hydrocarbons, such as dichloromethane, furthermore also alcohols, such as methanol, ethanol or isopropanol, and water. Mixtures of the above-mentioned solvents are furthermore suitable. TFA is preferably used in excess without addition of a further solvent, and perchloric acid is preferably used in the form of a mixture of acetic acid and 70% perchloric acid in the ratio 9:1. The reaction temperatures for the cleavage are advantageously between about 0 and about 50° C., preferably between 15 and 30° C. (room temperature).

The BOC, OtBu and Mtr groups can, for example, preferably be cleaved off using TFA in dichloromethane or using approximately 3 to 5N HCl in dioxane at 15-30° C., and the FMOC group can be cleaved off using an approximately 5 to 50% solution of dimethylamine, diethylamine or piperidine in DMF at 15-30° C.

Protecting groups which can be removed hydrogenolytically (for example CBZ, benzyl or the liberation of the amidino group from the oxadiazole derivative thereof) can be cleaved off, for example, by treatment with hydrogen in the presence of a catalyst (for example a noble-metal catalyst, such as palladium, advantageously on a support, such as carbon).

Suitable solvents here are those indicated above, in particular, for example, alcohols, such as methanol or ethanol, or amides, such as DMF. The hydrogeno lysis is generally carried out at temperatures between about 0 and 100° C. and pressures between about 1 and 200 bar, preferably at 20-30° C. and 1-10 bar. Hydrogeno lysis of the CBZ group succeeds well, for example, on 5 to 10% Pd/C in methanol or using ammonium formate (instead of hydrogen) on Pd/C in methanol/DMF at 20-30° C.

Examples of suitable inert solvents are hydrocarbons, such as hexane, petroleum ether, benzene, toluene or xylene; chlorinated hydrocarbons, such as trichloroethylene, 1,2- dichloroethane, tetrachloromethane, trifluoromethylbenzene, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether or ethylene glycol dimethyl ether (diglyme); ketones, such as acetone or butanone; amides, such as acetamide, dimethylacetamide, N-methylpyrrolidone (NMP) or dimethyl-formamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, or mixtures of the said solvents.

Esters can be hydrolyzed, for example, using HCl, H2SO4, or using LiOH, NaOH or KOH in water, water/THF, water/THF/ethanol or water/dioxane, at temperatures between 0 and 100° C.

Free amino groups can furthermore be acylated in a conventional manner using an acyl chloride or anhydride or alkylated using an unsubstituted or substituted alkyl halide, advantageously in an inert solvent, such as dichloromethane or THF and/or in the presence of a base, such as triethylamine or pyridine, at temperatures between −60° C. and +30° C.

For all the protection and deprotection methods, see Philip J . Kocienski, in “Protecting Groups”, Georg Thieme Verlag Stuttgart, New York, 1994 and, Theodora W. Greene and Peter G. M. Wuts in “Protective Groups in Organic Synthesis”, Wiley Interscience, 3^{rd} Edition 1999.

Reaction schemes as described in the example section are illustrative only and should not be construed as limiting the disclosure in any way.

### Synthesis of Formula (B) via Enzymatic Transformation Process

The disclosure also relates to synthesis of Formula (B). Formula (B) of the present disclosure comprise at least one sulfoxide. In some embodiments, the said sulfoxide is formed by the said enzymatic biotransformation process of this disclosure. In some embodiments, sulfur is a stereogenic (chiral) center.

This disclosure also provides a method for synthesis of diamine intermediate by an enzymatic biotransformation.

In some embodiments, compound of Formula (B) is at least 80%, at least 90%, at least 95%, at least 99%, at least 99.9% enantiomerically pure. In some embodiments enantiomerically pure compound of Formula (B) is ‘R’ isomer. In some embodiments, enantiomerically pure compound of Formula (B) is ‘S’ isomer.

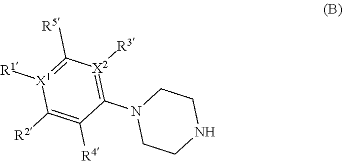

and salts and solvates thereof, wherein: X¹, X², R^{1′}, R^{2′}, R^{3′}, R^{4′}, R^{5′} are as defined above in formula (Ia).

According to one embodiment, the compounds of Formula (B) used or formed in/by the process of this disclosure are selected from the group consisting of:
(S)-1-(2-fluoro-4-(2-(methylsulfinyl)ethoxy)phenyl)piperazine;
(R)-1-(2-fluoro-4-(2-(methylsulfinyl)ethoxy)phenyl)piperazine;
(S)-1-(2,4-difluoro-5-(2-(methylsulfinyl)ethoxy)phenyl)piperazine; and
(R)-1-(2,4-difluoro-5-(2-(methylsulfinyl)ethoxy)phenyl)piperazine.

In some embodiments, the enzymatic biotransformation is in a cell free system.

In some embodiments, the enzymatic biotransformation comprises at least one enzyme selected from oxidoreductases. In some embodiments, the oxidoreductases selected from the group of monooxygenases and/or alcohol dehydrogenases. Preferably, alcohol dehydrogenase is selected from ethanol dehydrogenases. In some embodiments, wherein the enzymes are in crude or purified or in immobilized form.

In some embodiments, the oxidoreductases are derived from cyclohexanone monooxygenase from Arthrobacter sp., Pseudomonas sp., Rhodococcus jostii, Brachymonas petroleovorans, and/or Rhodococcus sp.

In some embodiments, enzymes are wild type enzymes. In some embodiments, enzymes are variants of wild type enzymes.

In some embodiment, the yield of enzymatic biotransformation process is at least 50%, at least 60%, at least 80%, at least 90%.

The enzymatic biotransformation for synthesis of compound of Formula (B) comprises at least two work-up steps. In some embodiments, work up steps can comprise denaturation, filtration, nano-filtration, degradation of sulfone, extraction, and recrystallization.

In some embodiments, the yield of the enzymatic biotransformation after work up steps is at least about 50%.

In some embodiments, the yield of the enzymatic biotransformation after work up steps is at least about 60%, is at least about 70%.

In some embodiments, the temperature of the enzymatic biotransformation process spans between about 0° C. and about 45° C.

In some embodiments, the solvent used for the enzymatic biotransformation comprises water and/or isopropanol.

In some embodiments, the enzymatic biotransformation comprises nicotinamide adenine dinucleotide phosphate (NADP+).

In some embodiments, oxygen content of the reactor where the enzymatic biotransformation is at least 0.1%, at least 0.3%, at least 0.5%, at least 1%, at least 5%, at least 7%, at least 10%, at least 15%.

In some embodiment, the enzymatic biotransformation comprises air flow. In some embodiments, the rate of air flow in 80 L reactor is between 0.5-1.5 L/min, 2.0˜2.5 L/min, 3.0˜3.5 L/min, 5.0˜7.5 L/min. One skilled in the art knows that air flow rate can be changed depending on the reaction conditions such as reactor volume.

In some embodiments, the enzymatic biotransformation comprises overoxidation of an undesired enantiomer, whereby undesired enantiomer is converted to a sulfone. In a preferred embodiment, the sulfone is removed by work up steps. In one embodiment, the work up step is recrystallization.

In some embodiments, the enzymatic biotransforation yields Intermediate B in at least about 85%, about 86%, about about 87%, about 88%, about 89%, about 90%, about 91%, about 92%, about 93%, about 94%, about 95%, about 96%, about 97%, about 98% ee, about 99% ee or about 99.9% ee. In some embodiments, the enzymatic biotransforation yields Intermediate B in at least about 85%. In some embodiments, the enzymatic biotransforation yields Intermediate B in at least about 90%. In some embodiments, the enzymatic biotransforation yields Intermediate B in at least about 95%. In some embodiments, the enzymatic biotransforation yields Intermediate B in at least about 96%. In some embodiments, the enzymatic biotransforation yields Intermediate B in at least about 97%. In some embodiments, the enzymatic biotransforation yields Intermediate B in at least about 98%. In some embodiments, the enzymatic biotransforation yields Intermediate B in at least about 99%. In some embodiments, the enzymatic biotransforation yields Intermediate B in at least about 99.5%. In some embodiments, the enzymatic biotransforation yields Intermediate B in at least about 99.9%.

The present disclosure includes enumerated embodiments 1-27:
1. An enzymatic biotransformation process for manufacturing compound of Formula (B) or a pharmaceutically acceptable salt or solvate thereof, wherein Formula (B) is at least 80%, at least 90%, at least 95%, at least 99%, at least 99.9% enantiomerically pure, wherein the enzymatic biotransformation comprises a solvent, and the temperature of the biotransformation is below the boiling temperature of the solvent

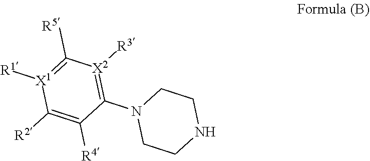

wherein
   X¹ and X² represent each independently C or N; R¹ is absent when X¹ is N; or when X¹ is C, R^{1′} represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino or alkylsulfonealkyl; said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylamino alkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;
   R^{2′} represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino, or alkylsulfonealkyl; said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)amino carbonyl, alkylamino alkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl; or R^{1′} and R^{2′} form together with the atoms to which they are attached a 5- or 6-membered aryl ring, a 5- or 6-membered heretoaryl ring, a 5- or 6-membered cycloalkyl ring or a 5- or 6-membered heretocyclyl ring; optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alky loxy carbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;
   wherein each of R^{1′} and R^{2′} comprises at least one sulfoxide as permitted by valency;
   R^{3′} is absent when X² is N; or when X² is C, R³ represents H or halo, preferably H or F;
   R^{4′} represents H or halo, preferably H or F; and R^{5′} represents H or halo, preferably H or F.
2. An enzymatic biotransformation process according to embodiment 1, wherein the compound is of formula (B-1) or a pharmaceutically acceptable salt or solvate thereof,

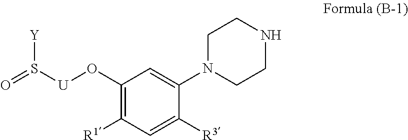

wherein
   R^{1′} and R^{3′} are as defined in embodiment 1;
   U represents alkylene, arylene, heteroarylene or heterocyclyene optionally substituted by one or more substituent selected from halo, hydroxy, alkyl, heterocyclylalkyl, hydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkoxy, amino, dialkylamino, aminoalkylcarbonylamino, aminocarbonylalkylamino, heterocyclylalkylaminocarbonyl, (aminocarbonylalkyl)(alkyl)amino, hydroxycarbonyl, aminocarbonyl, aminoalkylaminocarbonyl, alkylaminoalkylaminocarbonyl, dialkylaminoalkylaminocarbonyl, (alkylaminoalkyl)(alkyl)aminocarbonyl, heterocyclylcarbonyl, alkylsulfoxide and alkylsulfonealkyl;
   Y represents represents an alkyl, arylene, heteroarylene or heterocyclyene optionally substituted by one or more group selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;
   or U and Y form together with the atoms to which they are attached an aryl ring, a heteroaryl ring, a cycloalkyl ring or a heterocyclyl ring; optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.
3. An enzymatic biotransformation process according to embodiment 1, wherein the compound of Formula (B) is selected from the group consisting of:
(S)-1-(2-fluoro-4-(2-(methylsulfinyl)ethoxy)phenyl)piperazine;
(R)-1-(2-fluoro-4-(2-(methylsulfinyl)ethoxy)phenyl)piperazine;
(S)-1-(2,4-difluoro-5-(2-(methylsulfinyl)ethoxy)phenyl)piperazine; and
(R)-1-(2,4-difluoro-5-(2-(methylsulfinyl)ethoxy)phenyl)piperazine.
   4. A process for manufacturing a compound of Formula (Ia), or a pharmaceutically acceptable salt or solvate thereof,

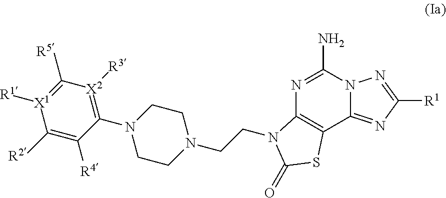

wherein
   R¹ represents 5- or 6-membered heteroaryl or 5- or 6-membered aryl, wherein heteroaryl or aryl groups are optionally substituted by one or more substituent selected from C1-C6 alkyl (preferably methyl) and halo (preferably fluoro or chloro); preferably R1 represents 5-membered heteroaryl; more preferably R1 represents furyl;
   X¹ and X² represent each independently C or N; R¹ is absent when X¹ is N; or when X¹ is C, R^{1′} represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino or alkylsulfonealkyl; said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylamino alkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;
   R^{2′} represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino, or alkylsulfonealkyl; said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)amino carbonyl, alkylamino alkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl; or R^{1′} and R^{2′} form together with the atoms to which they are attached a 5- or 6-membered aryl ring, a 5- or 6-membered heretoaryl ring, a 5- or 6-membered cycloalkyl ring or a 5- or 6-membered heretocyclyl ring; optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alky loxy carbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;
   wherein each of R¹′ and R²′ comprises at least one sulfoxide as permitted by valency
   R^{3′} is absent when X² is N; or when X² is C, R³ represents H or halo, preferably H or F;
   R^{4′} represents H or halo, preferably H or F; and R^{5′} represents H or halo, preferably H or F, comprising the steps of:
      (iii) Synthesis of diamine intermediate of Formula (B) by an enzymatic biotransformation, wherein the synthesis produces the intermediate of Formula (B) at least 80%, at least 90%, at least 95%, at least 99%, or at least 99.9% enantiomerically purity:

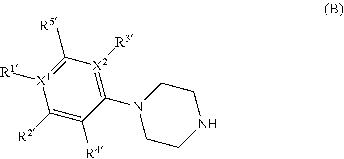

wherein, R^{1′}, R^{2′}, R^{3′}, R^{4′}, and R^{5′} are as defined above in this embodiment, the enzymatic biotransformation is performed in a solvent, and the temperature of the biotransformation is below the boiling temperature of the solvent;
   (iv) introduction of an intermediate of Formula (A) and the intermediate of Formula (B) into a suitable solvent:

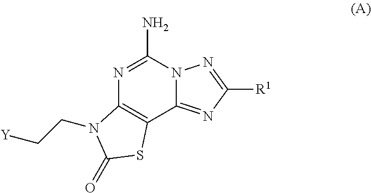

wherein, R¹ is as defined above in this embodiment and Y represents halo, alkylsulfonyloxy having 1 to 6 carbon atoms or arylsulfonyloxy having 6 to 10 carbon atoms; and
   (v) coupling between intermediate of Formula (B) and intermediate of Formula (A) in the suitable solvent at a temperature ranging from about 20° C. to about 180° C., thereby forming the compound of Formula (Ia).
5. The process according to embodiment 4, wherein the intermediate of Formula (B) is selected from the group consisting of:
(S)-1-(2-fluoro-4-(2-(methylsulfinyl)ethoxy)phenyl)piperazine;
(R)-1-(2-fluoro-4-(2-(methylsulfinyl)ethoxy)phenyl)piperazine;
(S)-1-(2,4-difluoro-5-(2-(methylsulfinyl)ethoxy)phenyl)piperazine; and
(R)-1-(2,4-difluoro-5-(2-(methylsulfinyl)ethoxy)phenyl)piperazine.
   6. The process according to embodiment 4, wherein the compound of Formula (Ia) is selected from the group consisting of: (S)-5 -amino-3 -(2-(4-(2-fluoro-4-(2-(methylsulfinyl)ethoxy)phenyl)piperazin-1-yl)ethyl)-8-(furan-2-yl)thiazolo [5,4-e][1,2,4]triazolo[1,5 -c]pyrimidin-2(3H)-one;
(R)-5-amino-3-(2-(4-(2-fluoro-4-(2-(methylsulfinyl)ethoxy)phenyl)-piperazin-1-yl)ethyl)-8-(furan-2-yl)thiazolo [5,4-e][1,2,4]triazolo [1,5 -c]pyrimidin-2(3H)-one;
(+)-(S)-5-amino-3-(2-(4-(2,4-difluoro-5-(2-(methylsulfinyl)ethoxy)phenyl)piperazin-1-yl)ethyl)-8-(furan-2-yl)thiazolo [5,4-e][1,2,4]triazolo[1,5-c]pyrimidin-2(3H)-one;
(−)-(R)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(S)-5-amino-8-(furan-2-yl)-3-(2-(4-(4-(methylsulfinyl)phenyl)piperazin-1-yl)ethyl)thiazolo[5,4-e][1,2,4]triazolo[1,5-c]pyrimidin-2(3H)-one;
(R)-5-amino-8-(furan-2-yl)-3-(2-(4-(4-(methylsulfinyl)phenyl)piperazin-1-yl)ethyl)thiazolo [5,4-e][1,2,4]triazolo[1,5-c]pyrimidin-2(3H)-one;
(S)-5-amino-3-(2-(4-(2,4-difluoro-5-(methylsulfinyl)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2,4-difluoro-5-(methylsulfinyl)phenyl)piperazin-1-yl)ethyl)-8-(furan-2-yl)thiazolo[5,4-e][1,2,4]triazolo[1,5-c]pyrimidin-2(3H)-one;
(S)-5-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-2,4-difluoro-N-(2-(methylsulfinyl)ethyl)benzamide;
(R)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-(2-(methylsulfinyl)ethyl)benzamide;
(S)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-methyl-N-(2-(methylsulfinyl)ethyl)benzamide;
(R)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-methyl-N-(2-(methylsulfinyl)ethyl)benzamide;
(R)-5-amino-3-(2-(4-(2-fluoro-4-(methylsulfinyl)phenyl)piperazin-1-yl)ethyl)-8-(furan-2-yl)thiazolo[5,4-e][1,2,4]triazolo[1,5-c]pyrimidin-2(3H)-one;
(S)-5-amino-3-(2-(4-(2-fluoro-4-(methylsulfinyl)phenyl)piperazin-1-yl)ethyl)-8-(furan-2-yl)thiazolo[5,4-e][1,2,4]triazolo[1,5-c]pyrimidin-2(3H)-one;
(S)-4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methylsulfinyl)ethyl)benzamide;
(R)-4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methylsulfinyl)ethyl)benzamide;
(S)-5-amino-3-(2-(4-(2,4-difluoro-5-(3-(methylsulfinyl)propoxy)phenyl)piperazin-1-yl)ethyl)-8-(furan-2-yl)thiazolo[5,4-e][1,2,4]triazolo[1,5-c]pyrimidin-2(3H)-one; and pharmaceutically acceptable salts or solvates thereof.
   7. The process according to any one of the embodiments 1-6, wherein the enzymatic biotransformation is in a cell free system.
   8. The process according to any one of the embodiments 1-6, the enzymatic biotransformation comprises at least one enzyme selected from oxidoreductases.
   9. The process according to embodiment 8, the oxidoreductases selected from the group of monooxygenases and/or alcohol dehydrogenases.
   10. The process according to embodiment 9, wherein the said monooxygenase is derived from Rhodococcus jostii.
   11. The process according to any one of the embodiments 9-10, wherein the said monooxygenase comprises a polypeptide sequence having at least 85%, 86%, 87%, 88%, 89%, 90%, 91%, 92%, 93%, 94%, 95%, 96%, 97%, 98%, 99%, or more sequence identity to SEQ ID NO:1.
   12. The process according to embodiment 9, wherein the said alcohol dehydrogenases comprises a polypeptide sequence having at least 85%, 86%, 87%, 88%, 89%, 90%, 91%, 92%, 93%, 94%, 95%, 96%, 97%, 98%, 99%, or more sequence identity to SEQ ID NO: 2.
   13. The process according to any one of the embodiments 1-12, wherein the enzymes are in crude or purified or in immobilized form.
   14. The process according any one of the embodiments 4-13, wherein the temperature in step (i) is between about 0° C. and about 45° C.

15. The process according to any one of the embodiments 4-14, wherein the solvent in step (i) comprises water and/or isopropanol.
16. The process according to any one of the embodiments 4-15, wherein the step (i) further comprises nicotinamide adenine dinucleotide phosphate (NADP⁺).
17. The process according to any one of the embodiments 4-16, wherein the step (i), (ii) and (iii) are performed in a reactor.
18. The process according to any one of the embodiments 4-16, wherein the reactor is different for each step (i), (ii) and (iii).
19. The process according to any one of the embodiments 1-18, enantiomerically pure compound of Formula (B) is ‘R’ isomer.
20. The process according to any one of the embodiments 1-18, enantiomerically pure compound of Formula (B) is ‘S’ isomer.
21. The process according to any one of the embodiments 1-20, the synthesis of intermediate of Formula (B) comprises a reaction yield.
22. The process according to embodiment 21, wherein the reaction yield is at least about 50%.
23. The process according to any one of the embodiments 1-22, wherein the synthesis of intermediate of Formula (B) comprises overoxidation of an undesired enantiomer, whereby undesired enantiomer is converted to a sulfone.
24. The process according to embodiment 23, wherein the sulfone is removed by recrystallization.
25. A process for the synthesis of a compound of formula (IV)

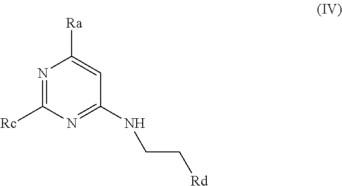

wherein, Ra is absent or is halo; Rb is halo; Rc is absent or is amino; Rd is absent or is methoxy; comprising the steps of: reacting the compound of formula (II)

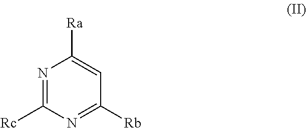

with a compound of formula (III)

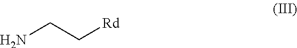

in the presence of a base.
26. A process for the synthesis of a compound of formula (V)

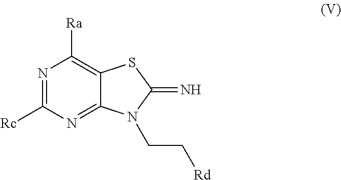

wherein, Ra is absent or is halo; Rc is absent or is amino; Rd is absent or is methoxy; comprising the steps of:
   (i) preparing the compound of formula (IV) according to the embodiment 25;
   (ii) reacting compound of formula (IV) with KSCN and Br₂ in the presence of an acid at a temperature below 0° C.;
   (iii) addition of a base selected from ammonia, barium hydroxide, calcium hydroxide, cesium hydroxide, magnesium hydroxide, potassium hydroxide, or sodium hydroxide to yield a compound of formula (V).
27. A process for the synthesis of a compound of formula (VI)

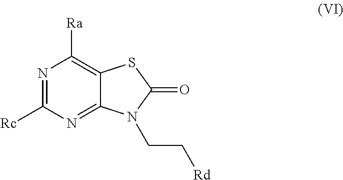

wherein, Ra is absent or is halo; Rc is absent or is amino; Rd is absent or is methoxy; comprising the steps of:
   (i) preparing the compound of formula (IV) according to the embodiment 26;
   (ii) dissolving the compound of formula (IV) in a water miscible organic solvent; and
   (iii) addition of an acid to yield a compound of formula (VI).

### BRIEF DESCRIPTION OF FIGURES

FIG. 1: Schematic representation of protocol for large scale synthesis of intermediate compound B by enzymatical transformation

### EXAMPLES

The present disclosure will be better understood with reference to the following examples. These examples are intended to representative of specific embodiments of the disclosure, and are not intended as limiting the scope of the disclosure.

The following abbreviations are used:
CHMO: monooxygenase enzyme
ADH: alcohol dehydrogenase enzyme
IPA: Isopropyl alcohol
DCM: Dichloromethane
Wt: weight percentage
DIPEA: N,N-Diisopropylethylamine, or Hünig's base
HPLC: High-Pressure Liquid Chromatography
GMP: Good Manufacturing Practice
ND: Not Detected

### Example 1

### Selection and Optimization of Enzymes

### 1.1 Enzyme Screening and Selection of Best Candidate

138 monooxygenases were screened to identify a suitable enzyme for the enzymatic synthesis of (+)-1-(2,4-difluoro-5-(2-(methylsulfinyl)ethoxy)phenyl)piperazine (compound 3a in Table 1) which is a key intermediate of a series of thiocarbamate derivatives. The enzyme screening tests for were conducted and eight enzymes derived from 5 different organisms shown in Table 2 were identified based on several different criteria such as enantiomeric excess (ee), conversion efficiency, and chemoselectivity (oxidation only at the desired site e.g. on sulfur atom). Among eight enzymes identified during screening process, enzyme 3 from Rhodococcus jostii showed highest ee value without obvious impurities such as sulfone formation; therefore, it was selected for further optimization. After optimization process, the intrinsic ee value of enzyme 3 was roughly 96%, but the ee value increased to >99% along with the overoxidation of opposite enantiomer (R).

Overall Synthetic Route for Enzymatic Synthesis of (+)-1-(2,4-difluoro-5-(2-(methylsulfinyl)ethoxy)phenyl)piperazine:

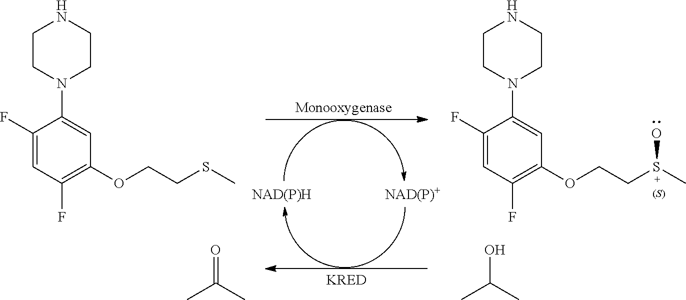

Reaction Conditions for Screening Process

Reaction Condition 1: 1-(2,4-difluoro-5-(2-(methylthio)ethoxy)phenyl)piperazine (20 mg, 0.07 mmol), isopropanol (100 μL, 10% v/v), NADP+ (2 mg, 0.0024 mmol), NAD+ (2 mg, 0.003 mmol), crude monooxygenase (14 mg enzyme powder) in buffer (200 mM, PB8.0), crude alcohol dehydrogenase (20 mg enzyme solution), the final volume was 1 mL, 30° C., 200 rpm.

TABLE 2

| | | | Product | | | |
|---|---|---|---|---|---|---|
| | | | Product | | Substrate | |
| | | | (0.568 | By- | (0.670 | |
| Enzyme | Enzyme Number | Source notes | min) | products | min) | ee % |
| cyclohexanone | Enzyme 1 | Wild type | 13.02 | 0 | 86.98 | 27.64 |
| monooxygenase from | | enzymes | | | | |
| Arthrobacter sp. | | | | | | |
| cyclopentadecanone 1,2- | Enzyme 2 | | 10.03 | 0 | 89.97 | −7.98 |
| monooxygenase from | | | | | | |
| Pseudomonas sp. | | | | | | |
| monooxygenase from | Enzyme 3 | | 6.95 | 0 | 93.05 | 72.08 |
| Rhodococcus jostii | | | | | | |
| cyclohexanone | Enzyme 4 | Variants | 45.28 | 0 | 54.72 | −93.74 |
| monooxygenase variant | | | | | | |
| from Brachymonas | Enzyme 5 | | 38.23 | 0 | 61.77 | −89.8 |
| petroleovorans | Enzyme 6 | | 35 | 0 | 65 | −89.94 |
| cyclohexanone | Enzyme 7 | Variants | 5.64 | 0 | 94.36 | 41.66 |
| monooxygenase variant | | | | | | |
| from Rhodococcus sp. | Enzyme 8 | | 5.38 | 0 | 94.62 | 19.76 |

### 1.2 Preliminary Reaction Optimization for Enzyme 3

Reaction Condition 2: 1-(2,4-difluoro-5-(2-(methylthio)ethoxy)phenyl)piperazine (20 mg, 0.07 mmol), isopropanol (100 μL, 10% v/v), PEG400 (100 μL), NADP+ (2 mg, 0.0024 mmol), NAD+ (2 mg, mmol), crude monooxygenase (20 mg enzyme powder) in buffer (200 mM, PB8.0), crude alcohol dehydrogenase (20 mg enzyme solution), the final volume was 2 mL, 30° C., 200 rpm.

Reaction Condition 3: 1-(2,4-difluoro-5-(2-(methylthio)ethoxy)phenyl)piperazine (20 mg, 0.07 mmol), isopropanol (100 μL, 5% v/v), Tween-80 (100 μL, 5% v/v), NADP+ (2 mg, 0.0024 mmol), crude monooxygenase (20 mg enzyme powder) in buffer (200 mM, PB8.0), crude alcohol dehydrogenase (20 mg enzyme solution), the final volume was 2 mL, 30° C., 200 rpm.

A preliminary reaction optimization was conducted to synthesize (+)-1-(2,4-difluoro-5-(2-(methylsulfinyl)ethoxy)phenyl)piperazine. Optimization conditions were summarized in Table 3. Lower IPA concentration (5%) was found to give better result. Adding 5% surfactant such as tween 80 was helpful for the reaction. The optimal cofactor was determined to be NADP+. With optimized reaction condition using enzyme 3, the conversion rate of starting material to (+)-1-(2,4-difluoro-5-(2-(methylsulfinyl)ethoxy)phenyl)piperazine reached 60.01% without obvious impurities, while the ee value reached 94.91% (Table 3).

TABLE 3

| | Product | | | |
|---|---|---|---|---|
| Reaction condition | Product | Impurity | Substrate | ee |
| Same as condition 2 | 11.17 | 0 | 88.83 | n. d. |
| Same as condition 2 | 9.46 | 0 | 90.54 | n. d. |
| Same as condition 2 | 10.20 | 0 | 89.80 | n. d. |
| Condition 2 and pH = 8.5 | 10.06 | 0 | 89.94 | n. d. |
| Condition 2 and pH = 9.0 | 3.02 | 0 | 96.98 | n. d. |
| Condition 2 and T = 25° C. | 7.27 | 0 | 92.73 | n. d. |
| Condition 2 and T = 35° C. | 3.26 | 0 | 96.74 | n. d. |
| Condition 2 and T = 40° C. | 3.29 | 0 | 96.71 | n. d. |
| Condition 2 and IPA = 5% | 16.43 | 0 | 83.57 | n. d. |
| Condition 2 and | 14.12 | 0 | 85.88 | n. d. |
| IPA = 7.5% | | | | |
| Condition 2 and | 9.28 | 0 | 90.72 | n. d. |
| IPA = 12.5% | | | | |
| Condition 2 and add 5% | 8.27 | 0 | 91.73 | n. d. |
| methanol | | | | |
| Condition 2 and add 5% | 11.85 | 0 | 88.15 | n. d. |
| PEG 400 | | | | |
| Condition 2 and add 5% | 21.20 | 0 | 78.80 | n. d. |
| Tween-80 | | | | |
| Condition 2 and add 5% | 18.59 | 0 | 81.41 | n. d. |
| Triton-100 | | | | |
| Condition 2 and add 5% | 2.11 | 0 | 97.89 | n. d. |
| MTBE | | | | |
| Condition 2 without NAD⁺ | 13.75 | 0 | 86.25 | n. d. |
| Condition 2 without | 3.27 | 0 | 96.73 | n. d. |
| NADP⁺ | | | | |
| Same as condition 3 | 0 | 0 | 98.46 | n. d. |
| Same as condition 3 | 60.01 | 0 | 39.99 | 94.91 |
| Same as condition 3 | 26.68 | 69.30 | 4.02 | 85.77 |
| Same as condition 3 | 0 | 100 | 0 | n. d. |
| Same as condition 3 | 59.31 | 39.50 | 1.19 | 69.46 |
| Same as condition 3 | 78.47 | 16.00 | 5.53 | 62.64 |

n. d. = not determined

### 1.3 Further Reaction Optimization for Enzyme 3

Enzymatic catalytic reaction was further optimized by altering quantity of enzyme and of cosolvent and volume of reaction relative to substrate amount.

Lower IPA concentration (2%) was found to give better result. Adding 15% surfactant such as tween-80 was helpful for the reaction. The optimum ADH dosage was determined to be 0.5 wt. Then, the reaction was amplified up to 100 mg scale, 2 wt CHMO gave the best result, with a residual substrate 1.56% and ee value>99% (Table 4). The intrinsic ee value was roughly estimated as 96% (Conv.=70.73%, ee=96.02%), but the ee value increased along with the overoxidation of opposite enantiomer (R). Then, the reaction volume was reduced to 60 V, 4 wt enzyme loading gave a good result with a residual substrate 1.12%, and the ee value>99% (Table 4).

Reaction Condition 4: 1-(2,4-difluoro-5-(2-(methylthio)ethoxy)phenyl)piperazine (20 mg, 0.07 mmol), isopropanol (100 μL, 5% v/v), Tween-80 (100 μL, 5% v/v), NADP+ (4 mg, 0.0024 mmol), crude monooxygenase solution (10 wt) in buffer (200 mM, PB8.0), crude alcohol dehydrogenase solution (2 wt), the final volume was 2 mL, 30° C., 200 rpm.

Reaction Condition 5: 1-(2,4-difluoro-5-(2-(methylthio)ethoxy)phenyl)piperazine (100 mg, 0.35 mmol), isopropanol (0.2 mL, 2% v/v), Tween-80 (1.5 mL, 15% v/v), NADP+ (20 mg, 0.012 mmol), crude monooxygenase solution (8 wt) in buffer (200 mM, PB8.0), crude alcohol dehydrogenase solution (0.5 wt), 200 rpm.

TABLE 4

| | Product | | | |
|---|---|---|---|---|
| Reaction condition | Product | Impurity | Substrate | ee |
| Same as condition 4 | 50.35 | 0 | 49.65 | n. d. |
| CHMO (10 wt) | | | | |
| Same as condition 4 | 40.81 | 0 | 59.19 | n. d. |
| CHMO (8 wt) | | | | |
| Same as condition 4 | 30.89 | 0 | 69.11 | n. d. |
| CHMO (6 wt) | | | | |
| Same as condition 4 | 20.68 | 0 | 79.32 | n. d. |
| CHMO (4 wt) | | | | |
| Same as condition 4 | 17.68 | 0 | 82.32 | n. d. |
| CHMO (3 wt) | | | | |
| Same as condition 4 | 10.96 | 0 | 89.04 | n. d. |
| CHMO (2 wt) | | | | |
| Same as condition 4 | 5.44 | 0 | 94.56 | n. d. |
| CHMO (1 wt) | | | | |
| Same as condition 4 | 31.55 | 0.04 | 68.40 | n. d. |
| ADH (2 wt) | | | | |
| Same as condition 4 | 32.96 | 0.09 | 66.95 | n. d. |
| ADH (1 wt) | | | | |
| Same as condition 4 | 38.17 | 0.08 | 61.75 | n. d. |
| ADH (0.5 wt) | | | | |
| Same as condition 4 | 34.52 | 0.06 | 65.41 | n. d. |
| ADH (0.3 wt) | | | | |
| Same as condition 4 | 41.36 | 0.11 | 58.53 | n. d. |
| Tween-80 (100 μL) | | | | |
| Same as condition 4 | 70.10 | 1.0 | 28.90 | 96.88 |
| Tween-80 (200 μL) | | | | |
| Same as condition 4 | 78.88 | 1.83 | 19.30 | 98.13 |
| Tween-80 (300 μL) | | | | |
| Same as condition 4 | 92.01 | 5.90 | 2.09 | >99 |
| IPA (40 μL) | | | | |
| Same as condition 4 | 90.45 | 2.12 | 7.43 | 98.18 |
| IPA (60 μL) | | | | |
| Same as condition 4 | 94.12 | 3.31 | 2.56 | >99 |
| IPA (80 μL) | | | | |
| Same as condition 4 | 70.73 | 1.07 | 28.20 | 96.02 |
| IPA (100 μL) | | | | |
| Same as condition 5 | 74.87 | 24.72 | 0.41 | >99 |
| CHMO (8 wt) | | | | |
| Same as condition 5 | 79.71 | 19.80 | 0.49 | >99 |
| CHMO (6 wt) | | | | |
| Same as condition 5 | 89.97 | 10.54 | 0.50 | >99 |
| CHMO (4 wt) | | | | |
| Same as condition 5 | 94.08 | 4.36 | 1.56 | >99 |
| CHMO (2 wt) | | | | |
| Same as condition 5 | 73.18 | 26.62 | 0.20 | >99 |
| Volume (100 V) | | | | |
| Same as condition 5 | 75.45 | 24.34 | 0.22 | >99 |
| Volume (80 V) | | | | |
| Same as condition 5 | 88.27 | 11.56 | 0.17 | >99 |
| Volume (60 V) | | | | |
| Same as condition 5 | 82.02 | 15.05 | 0.63 | >99 |
| Volume (60 V, 8 wt) | | | | |
| Same as condition 5 | 86.68 | 9.25 | 0.17 | >99 |
| Volume (60 V, 6 wt) | | | | |
| Same as condition 5 | 93.31 | 4.36 | 1.12 | >99 |
| Volume (60 V, 4 wt) | | | | |
| Same as condition 5 | 76.10 | 1.51 | 21.91 | n. d. |
| Volume (40 V, 8 wt) | | | | |
| Same as condition 5 | 58.92 | 0.73 | 39.98 | n. d. |
| Volume (40 V, 6 wt) | | | | |
| Same as condition 5 | 50.36 | 0.35 | 49.28 | n. d. |
| Volume (40 V, 4 wt) | | | | |

n. d. = not determined

After screening and validation, enzyme 3 (Table 7, Accession number: ABG97104.1) were selected to be used in a large scale synthesis of (+)-1-(2,4-difluoro-5-(2-(methylsulfinyl)ethoxy)phenyl)piperazine. On large scale, synthesis parameters such as airflow, stirrer rate etc. were changed. Additionally, enzyme 3 catalyzed the conversion of sulfoxide to sulfone impurity which is a good way to improve stereoselectivity by over oxidizing the other enantiomer (R) and then removing the sulfone impurity from sulfoxide product without losing yield.

### Example 2

### Large-Scale Synthesis of Compound 3A

### 2.1 Synthesis of Intermediate A

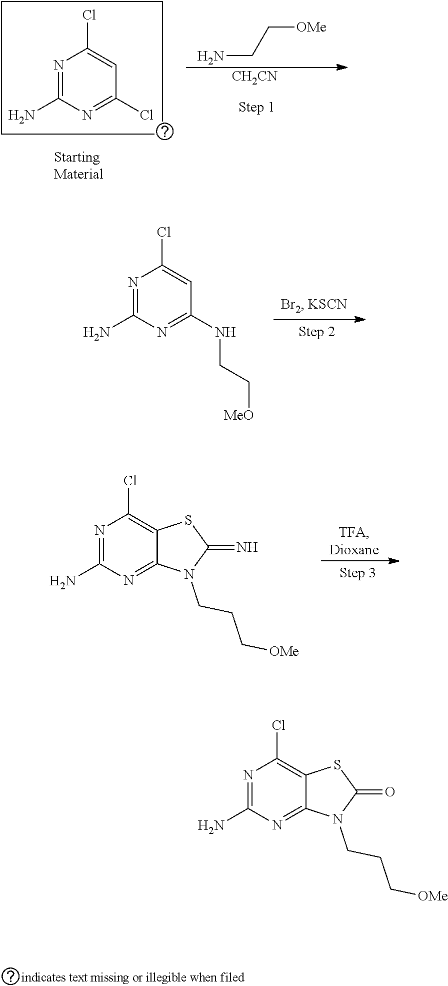

### Step 1:

4,6-dichloropyrimidin-2-amine (82.0 kg, 1 wt, 1.0 eq) and Acetonitrile (660 kg, 8.05 wt) were charged to the reactor, then 2-methoxyethan-1-amine (57.0 kg, 0.70 wt, 1.5 eq) and DIPEA (78.8 kg, 0.96 wt, 1.2 eq) were added. The temperature was adjusted to 55-65° C. and the reaction was stirred until it was completed. The reaction end point was checked by HPLC. The reaction mixture was cooled to 5-15° C. over 6 h and stirred for 2 h. The solids were isolated by centrifuge and the wet cake was washed with Acetonitrile (150.0 kg, 1.83 wt). The wet cake was dried at 45-55° C. to give 6-chloro-N4-(2-methoxyethyl)pyrimidine-2,4-diamine (92.25kg, 91.1% yield).

### Step 2:

KSCN (87.0 kg, 1.05 wt, 2.2 eq) and propionic acid (497 kg, 5.99 wt) were charged to the reactor and the temperature was adjusted to between minus 25° C. to minus 10° C. (−25 to −10° C.). Br₂ was added (0.88 wt, 1.1 eq) into the reactor maintaining a temperature below minus 10° C. (−10° C.).

A filtered solution of 6-chloro-N4-(2-methoxyethyl) pyrimidine-2,4-diamine (83.0 kg, 1 wt, 1.0 eq) were dissolved in propionic acid (332.8 kg, 3.85 wt) and added to the reactor while maintaining the reactor contents below minus 5° C. (−5° C.). The reaction was stirred at minus 15 to minus 5° C. (−15 to −5° C.) until the reaction is complete. Aqueous ammonia (−25% w/w, 917 kg, 11.05 wt) were then added while maintaining the reactor contents below 0° C. Once the addition was complete, the reaction temperature was adjusted to 45-55° C. and the reaction was stirred until the reaction is complete (Confirmed by HPLC). The reaction temperature was adjusted to 15-25° C. over 2 h and it was stirred for further 2 h. The solids were then separated by centrifuge and the wet cake was transferred into another reactor. Process water (498 kg, 6.00 wt) was added into the reactor and the temperature was adjusted to 15-25° C., then the reaction was stirred for 4 h. The wet cake was washed with H₂O (321.2 kg, 3.87 wt) and then it was dried at 50-70° C. to obtain the final product of step 2 (90.4 Kg, 85% yield).

### Step 3:

The final product of step 2 was charged into the reactor C19031849-B (90.4 kg, 1 wt, 1.0 eq) and dioxane (2,189 kg, 24.19 wt) was added and it was stirred at 20-40° C. for 3 h. The reaction was then filtered and washed with dioxane (101 kg, 1.12 wt). The reaction was transferred into another reactor, and H2O (95 kg, 1.05 wt) and TFA (99.0 kg, 1.09 wt, 2.5 eq) was added. The temperature as adjusted to 90-100° C. and stirred until the reaction was completed (Confirmed by HPLC). The reaction was concentrated to 5-7V below 55° C. The temperature was adjusted to 45-55° C. and H2O (900 kg, 9.94 wt) was added at 45-55° C. The temperature then adjusted to 55-65° C. and it was stirred over the course of 2 hours. Then, the reaction temperature was adjusted to 5-15° C. and stirred for 6 h. Solids were isolated by centrifuge, and the wet cake was washed with water (509 kg, 5.62 wt). The wet cake then was dried at 45-55° C. to give final product of step 3 (80.32 kg, 88.5% yield).

### Step 4:

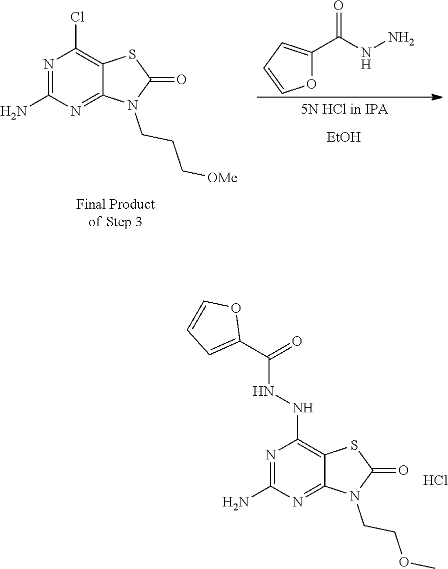 [SMILES: "EtOH" ; COCCCn1c(=O)sc2c(Cl)nc(N)nc21 ; NNC(=O)c1ccco1 ; COCCn1c(=O)sc2c(NNC(=O)c3ccco3)nc(N)nc21 ; Cl]

Final product of step 3 (37.3 Kg, 1.00 eq) and ethanol (20 rel. vol) were added to the reactor and the reactor was heated and stirred at 75-85° C., then the reaction was cooled to 20-30° C. and 2-furoic acid hydrazide (27.1 Kg, 1.5 eq) were added. After 10-15 minutes of stirring, 5-6 N HCl in 2-propanol (78 Kg, 3.0 eq) was added and the reactor contents were again heated to 75-85° C. The reaction was stirred for at least 18 hours until the reaction is complete (Confirmed by HPLC). Then, it was cooled to 20-30° C. and it was stirred for 2 hours. The solids were isolated by filtration and the isolated solids were washed with Methyl tert-Butyl Ether (110 Kg, 4.0 rel. vol). The solids were dried at 20-30° C. to give final product of step 4. (40.8 Kg, 79.8% yield)

### Step 5 & 6:

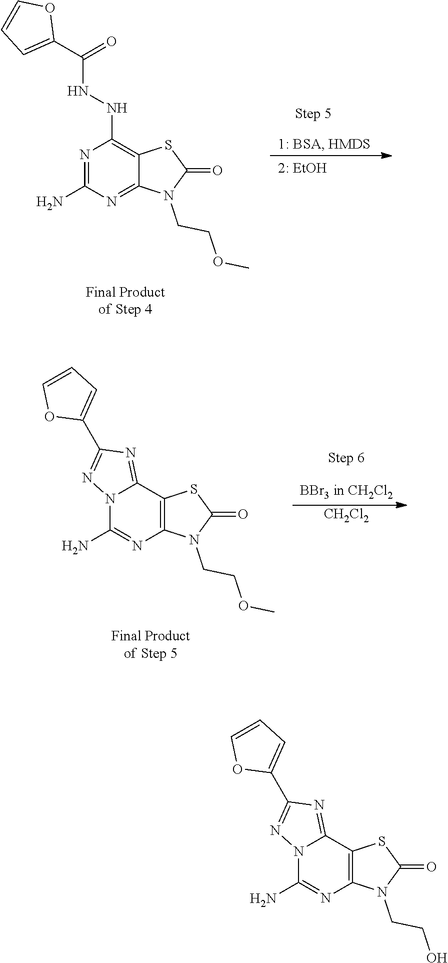 [SMILES: COCCn1c(=O)sc2c(NNC(=O)c3ccco3)nc(N)nc21 ; COCCn1c(=O)sc2c1nc(N)n1nc(-c3ccco3)nc21 ; "CH2Cl2" ; Nc1nc2c(sc(=O)n2CCO)c2nc(-c3ccco3)nn12]

### Step 5:

The final product of step 4 (40.8 Kg, 1.00 eq), Hexamethyldisilazane (314 Kg, 10 rel. vol) and N,O-Bis(trimethylsilyl)acetamide (170.4 Kg, 5.0 rel. vol) were added to the reactor and heated to 115-125° C. over 2 hours while stirring. Then the reaction was allowed to be stirred for 20 hours sampling to test conversion. When reaction was complete, it was cooled to 45-55° C. Dose ethanol, abs. (325.8 Kg, 10.0 rel. vol) was added to the reactor at 45-55° C. over 2 hours and stirred for 1 hour, then the reaction was cooled to 15-25° C. The solids were isolated by filtration and washed with ethanol 70%. Finally, the solid product was dried at 20-30° C. to give final product of step 5 (24.9 Kg, 84.6% yield).

### Step 6:

The final product of step 5 (24.9 Kg 1.00 eq) and dichloromethane (661 Kg, 20 rel. vol) were added to the reactor and stirred at 30-35° C. Dose boron tribromide 1.0 M in dichloromethane (329 Kg, 3.00 eq) was added at 30-35° C. over 1-2 h, then allowed stirring for at least 4.5 hours at 35-40° C. Then, the reaction was cooled to 20-30° C. The reactor temperature then was adjusted to 25-30° C. and dose methanol (15 rel. vol) was at 25-30° C. over 3 hours. The reactor was again heated to 35-45° C. and it was distilled off approximately 20 relative volumes solvent at reflux until 25 relative volumes remain.

Methanol (15 rel. vol) was added to the reactor and the reactor was heated to 40-50° C., then distilled off approximately 15 relative volumes solvent at reflux until 25 relative volumes remain.

Purified water (20 rel. vol) was added at 40-45° C. and distiled off approximately 25 relative volumes solvent at 30-50° C. and reduced pressure until 20 relative volumes remain.

Then, the reactor was cooled to 15-25° C. and pH was adjusted to 11±0.5 pH using 2 M sodium hydroxide while keeping the temperature at 20-30° C., then it was stirred for 1 hour.

The solids were isolated by filtration and the solids were slurry washed on the filter with purified water (4.0 rel. vol) at 15-25° C. for 15-20 mins, after removing the filtrate repeat the slurry wash a further three times.

The solids were slurry washed on the filter with methanol (4.0 rel. vol) at 15-25° C. for 15-20 mins, after removing the filtrate repeat the slurry wash once more. Dry the remaining solids at 35-45° C. to give final product of step 6 (21.3Kg, 89.3%).

### Step 7:

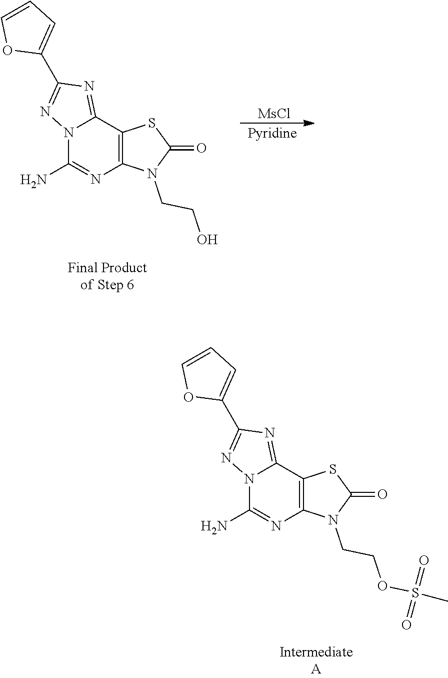 [SMILES: "MsCl" ; Nc1nc2c(sc(=O)n2CCO)c2nc(-c3ccco3)nn12 ; "Pyridine" ; CS(=O)(=O)OCCn1c(=O)sc2c1nc(N)n1nc(-c3ccco3)nc21]

Final product of step 6 (20.8 Kg, 1.00 eq) and pyridine (104 Kg, 5.0 rel. vol) were stirred at 15-25° C. Then, dose methanesulfonyl chloride (9.14 Kg, 1.2 eq) was added to the reactor at 15-25° C. over 1 hour, then the reaction was stirred for at least 6 hours at 15-25° C., monitoring conversion by HPLC. When the reaction was complete, dose purified water (211 Kg, 10 rel. vol) was added to the reactor at 15-25° C. over 1.5±0.5 hours and stirred for 1.5±0.5 hours.

The solids were isolated by filtration and it was washed three times with purified water (each 84 Kg, 4.0 rel. vol), then washed three times with tetrahydrofuran (each 75 Kg, 4.0 rel. vol).

The solids were dried at 15-25° C. for 12 hours to give intermediate A (23.2 Kg, 89.7% yield).

### 2.2 Synthesis of Intermediate (B) Using Enzymatic Transformation

### 2.2.1 Synthesis of Starting Material for Enzymatic Transformation

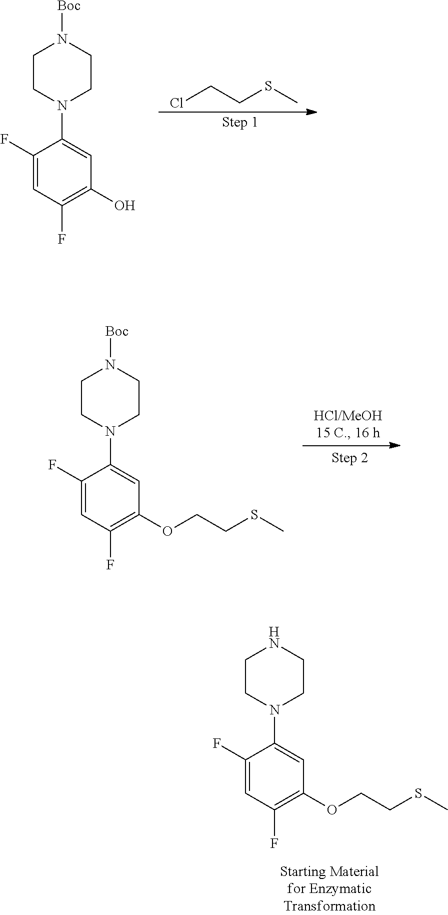

### Step 1:

Tert-butyl 4-(2,4-difluoro-5-hydroxyphenyl)piperazine-1-carboxylate (134.5 Kg, 1.00 eq.) and acetonitrile (1,085 Kg, 8.0 rel. vol) were added to the reactor and stirred. Then, K2CO3 (94.2 Kg, 1.6 eq.) were added and continued to stir for 30 min at 20-30° C. Then, 1-chloro-2-methylsulfanyl-ethane (51.7 Kg, 1.1 eq.) were added to the reaction and the temperature was adjusted to 78-84° C. and stirred for 8-10 hours. After 8-10 hours, the temperature was cooled to 20-30° C. and the reaction was checked for completion (by HPLC). The solids were removed by filtration, and washed with acetonitrile (165 Kg). Then, the filtrate and washings were combined in another reactor, then concentrate to 270-540 L at ≤50° C. under reduced pressure, then n-heptane (1,598 Kg) was charged into the reactor. The resulting solids were isolated by centrifuge to give tert-butyl 4-(2,4-difluoro-5-(2-(methylthio)ethoxy)-phenyl)piperazine-1-carboxylate (135.6 Kg, 82% yield)—the final product of step 1.

### Step 2:

Tert-butyl 4-(2,4-difluoro-5-(2-(methylthio)ethoxy)-phenyl)piperazine-1-carboxylate (91 Kg, 1.00 eq.) and methanol (364 Kg, 4.0 rel. vol) was added to the reactor and stirred for 15-30 mins while cooling to 5-15° C. slowly. Dose 4 M HCl/MeOH solution (100 kg) were added with stirring while maintaining the temperature at 5-15° C., then reaction was stirred at this temperature for a further 20-24 hours. Once reaction was complete (confirmed by HPLC), 9% NaOH solution (355 Kg) were slowly added in portions at ≤25° C. The mixture was concentrated at ≤45° C. under vacuum to 315-405 L and the aqueous layer was extracted twice with DCM (2×400 Kg) and the organic layers were combined in a clean reactor. N-heptane (1,077 Kg) was added and the mixture was concentrated at ≤45° C. under vacuum to 450-540 L. The solids were isolated by centrifuge and washed with n-heptane (46 Kg), dry at 50-60° C. for 18-25 h to give 1-(2,4-difluoro-5-(2-(methylthio)ethoxy)phenyl)piperazine (51.5 Kg, 79% yield)—starting material for enzymatic reaction.

### 2.2.2 Synthesis of Intermediate (B) by Enzymatic Transformation

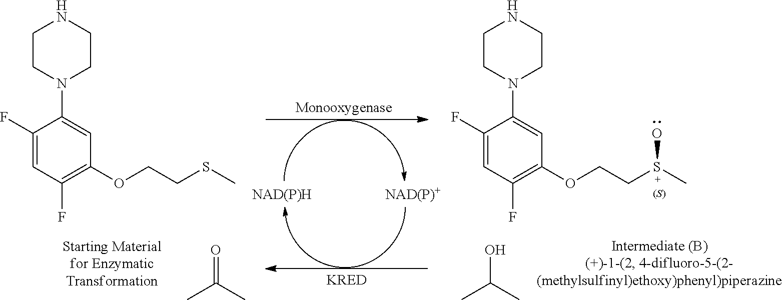

29.5 kg 1-(2,4-difluoro-5-(2-(methylsulfinyl)ethoxy)phenyl)piperazine-intermediate B was manufactured under GMP conditions using above illustrated biotransformation synthetic route with using monooxygenase enzyme 3 (Table 7, Accession number: ABG97104.1) and KRED (Enzyme 9, Table 7, Accession number: CAA46053.1) 49.7 kg starting material was used. After the work up process, intermediate B was obtained in 56.2% yield with 98.6% LCAP (Liquid Chromatography Area Percent), 97.4% assay and 100.0% ee. Summary of the reaction yield can be found in Table 5. Protocol for the procedure is summarized in Table 6.

TABLE 5

| Scale | Starting | | | | | Crude |
|---|---|---|---|---|---|---|
| (Reactor | Material, | Intermediate B, kg | | Purity | Assay | Yield |
| Size), L | kg | Theoretical | Actual | (%) | (%) | (%) |
| 8000 | 49.7 | 52.5 | 29.5, 28.7 | 98.6 | 97.4 | 56.2 |
| | | | corrected | | | |

Procedure (In 8000 L Reactor)

TABLE 6

| Procedure | Note | |
|---|---|---|
| 1. Exchange | | |
| A clean and dry 8000 L stainless steel reactor was | Oxygen content | 0.8% |
| evacuated to P ≤ −0.07 MPa then filled with N2 to | | |
| normal pressure. This operation was repeated for 3 | | |
| times until oxygen content was ≤1.0%. Dissolved | | |
| oxygen electrode calibration was corrected. | | |
| 2. Charging | | |
| Purified water (2011.9 kg), disodium phosphate | Charging temperature | 23.8~25.0° C. |
| dodecahydrate (82.8 kg), monosodium phosphate | Purified water | 2638.6 kg |
| (3.4 kg) were added into above reactor at 15~30° C. | Monosodium phosphate | 82.8 kg |
| The solid addition funnel and pipe were rinsed with | Disodium phosphate dodecahydrate | 3.4 kg |
| purified water (626.7 kg), the mixture was stirred for | Stirring time | 34 min |
| 0.5~1 h until the solid dissolved completely by | pH | 8.2 |
| visual check. The mixture was sampled for pH | | |
| analysis to make sure pH = 7.7~8.3. | | |
| 3. Adding | | |
| Isopropanol (78.7 kg) and starting material for | Adding temperature | 23.7~25.1° C. |
| enzymatic reaction (SME) (49.7 kg) were added into | Isopropanol | 78.7 kg |
| mixture at 15~30° C.The mixture was stirred for | SME | 49.7 kg |
| 10~20 min. The mixture was sampled for pH | Stirring time | 15 min |
| analysis to make sure pH = 8.0~9.0. | pH | 8.2 |
| 4. Adjusting temperature | | |
| The mixture was adjusted to 15~20° C. | Temperature | 20.0° C. |
| 5. Adding | | |
| While maintaining the temperature at 10~20° C., | Adding temperature | 17.3~17.9° C. |
| nicotinamide adenine dinucleotide phosphate | Nicotinamide adenine | 1.0 kg |
| (1.0 kg), monooxygenase-Enzyme 3 (198.3 kg) and | dinucleotide phosphate | |
| ethanol dehydrogenase-Enzyme 9 (24.6 kg) were | Monooxygenase-Enzyme 3 | 198.3 kg |
| added into mixture. | Ethanol dehydrogenase-Enzyme 9 | 24.6 kg |
| 6. Ventilation | | |
| Air was ventilated into mixture at a reference rate of | | |
| 3.0~5.0 m 3/h. Then nitrogen was ventilated into | | |
| mixture at a reference rate of 6.0~10.0 m 3/h. | | |
| 7. Reaction | | |
| The mixture was stirred for reaction at 15~20° C. The | Reaction temperature | 17.3~19.0° C. |
| dissolved oxygen value of the mixture was recorded | Reaction time | 34 h 30 min |
| every 1 h. 2 h later, the mixture was sampled for | Monooxygenase-Enzyme 3 | 18.4 kg |
| HPLC analysis every 1~3 h until area % of | SME | 1.3% |
| SME ≤1.5%, Sulfone ≤5.0% and ee ≥99.0%. | Sulfone | 2.8% |
| The SME >1.5 area % and difference between two | ee | 100.0% |
| consecutive samples was ≤0.5%, then | | |
| monooxygenase 507047 (18.4 kg) was added and | | |
| continued to react until area % of SME ≤1.5%, | | |
| Sulfone ≤5.0% and ee >99.0%. | | |

Sampling method: Take 5 ml mixture, quench into
10 ml mixture of acetonitrile and purified water (The
volume ratio is 1:1), the resulting homogeneous
mixture was submitted for test.

| | | |
|---|---|---|
| 8. Adjusting pH | | |
| While maintaining the temperature at 10~25° C., | Temperature | 18.9~19.2° C. |
| hydrochloric acid (88.4 kg) was added into mixture | Hydrochloric acid | 88.4 kg |
| at a rate of 30~60 kg/h. After adding, the mixture | pH | 2 |
| was sampled for pH analysis. | | |
| 9. Adding & Filtration | | |
| Celite (25.0 kg) was added into mixture, and stirred | Celite | 25.0 kg |
| for 1~2 h. Then the mixture was filtered with a | Stirring time | 1 h 1 min |
| plastic-lined centrifuge. The filtrate was discharged | Centrifugation time | 88 h 30 min |
| to a 5000 L reactor through in-line filter. | | |
| 10. Nanofiltration | | |
| The nanofiltration parameter was set as below: | Pump frequency | 30.9~34.4 Hz |
| Size of membrane: 5000 Da; Pump frequency: | Membrane circulation flow | 0.868~1.008 m 3/h |
| 30~35 Hz; membrane pressure: 0.5~0.9 MPa. Then | Membrane pressure | 5.2~9.0 bar |
| the mixture was concentrated until 100~200 kg left. | | |
| Purified water by two batches (126.4 kg + 141.4 kg) | Purified water | 267.8 kg |
| was added into the retentate and then continue to | Protein content | <50 ppm |
| concentrate. The permeates were combined and | Nanofiltration time | 137 h |
| sampled for protein content analysis to make sure it | | |
| was ≤50 ppm. | | |
| 11. Adjusting temperature | | |
| The mixture was adjusted to 20~35° C. under stirring. | Temperature | 23.4° C. |
| 12. Adding | | |
| While maintaining the temperature at 20~35° C., L- | Temperature | 23.4~27.9° C. |
| cysteine (10.0 kg) and anhydrous sodium carbonate | L- cysteine | 10.0 kg |
| (361.1 kg) was added into mixture. | Anhydrous sodium carbonate | 361.1 kg |
| 13. Decomposition of Sulfone | | |
| The mixture was stirred for reaction at 20~35° C. 2 h | Reaction temperature | 27.5~28.0° C. |
| later, the mixture was sampled for HPLC analysis | Reaction time | 6 h 30 min |
| every 1~3 h until area% of Sulfone was ≤0.2%. | Area % of Sulfone | ND |
| Sampling method: Take 5 ml mixture and submit for | | |
| analysis testing. | | |
| 14. Extraction | | |
| While maintaining the temperature at 20~30° C., the | Isopropyl acetate | 436.3 kg |
| mixture was extracted with isopropyl acetate by two | 1st Stirring time | 21 min |
| times (217.6 kg + 218.7 kg). The mixture was stirred | 1st Settling time | 53 min |
| for 15~30 min and then settled before separation. | 2nd Stirring time | 30 min |
| The aqueous phase was sampled for purity to make | 2nd Settling time | 3 h 10 min |
| sure SME ≤0.2 area %. | Area % of SME | ND |
| 15. Adding | | |
| While maintaining the temperature at 20~30° C., | Adding temperature | 20.2~24.5° C. |
| anhydrous sodium carbonate (360.4 kg) was added | Anhydrous sodium carbonate | 360.4 kg |
| portion-wise into aqueous phase at the interval of | Stirring time | 38 min |
| 30~60 min, the mixture was stirred for 0.5~1 h. | | |
| 16. Extraction | | |
| While maintaining the temperature at 20~30° C., the | Temperature | 22.6~24.2° C. |
| mixture was extracted with DCM five times | DCM | 1247.6 kg |
| (249.9 kg + 250.0 kg + 249.3 kg + 249.1 kg + 249.3 kg). | 1st Stirring time | 17 min |
| the mixture was stirred for 15~20 min every time | 1st Settling time | 45 min |
| and then settled before separation. | 2nd Stirring time | 20 min |
| | 2nd Settling time | 45 min |
| | 3rd Stirring time | 18 min |
| | 3rd Settling time | 2 h 44 min |
| | 4th Stirring time | 17 min |
| | 4th Settling time | 2 h 43 min |
| | 5th Stirring time | 20 min |
| | 5th Settling time | 2 h 25 min |
| 17. Washing | | |
| Sodium carbonate solution which was prepared with | Temperature | 13.9~15.2° C. |
| sodium carbonate (19.3 kg + 19.2 kg) and purified | Sodium carbonate | 38.5 kg |
| water (124.8 kg + 122.1 kg) was added into organic | Purified water | 246.9 kg |
| phase at 10~30° C. The organic phase was sampled | | |
| for HPLC analysis to make sure that the sample is | | |
| free from large quantity of sulfone degradation | | |
| products and other impurities. | | |
| 18. Concentration | | |
| The mixture was concentrated at Tjacket ≤45° C. | Jacket temperature | 35.6~39.2° C. |
| under reduced pressure (P ≤ −0.05 MPa) until | Concentration pressure | −0.09~0.08 MPa |
| 175~240 L left. | | |
| 19. Concentration | | |
| The mixture was transfer to 2000 L reactor through | Isopropyl acetate | 611.3 kg |
| in-line filter. Isopropyl acetate two | Concentration temperature | 19.4~33.6° C. |
| times(305.8 kg + 305.5 kg) was added into mixture | Concentration pressure | −0.096~0.080 MPa |
| through in-line filter and stirred for 10~15 min. Then | DCM residual | 0.5% |
| the mixture was concentrated at ≤45° C. under | | |
| reduced pressure (P ≤−0.08 MPa) until 170~200 L left. | | |
| The mixture was sampled for DCM residual to make | | |
| sure DCM ≤1.0% | | |
| 20. Cooling& Maintaining | | |
| The mixture was cooled to 15~20° C at a reference | Maintaining temperature | 18.5~19.0° C. |
| rate of 10~15° C., and then it was maintained and | Maintaining time | 50 min |
| stirred for 0.5~1 h at 15~20° C. | | |
| 21. Adding | | |
| n-Heptane (117.0 kg) was added into mixture | n-Heptane | 117.0 kg |
| through in-line filter at a reference rate of | | |
| 30~40 kg/h. | | |
| 22. Crystallization | | |
| The mixture was cooled to 0~10° C and maintained | Cooling temperature | 9.4° C |
| for crystallization. After 2 h, the mixture was | Crystallization temperature | 4.7~9.4° C |
| sampled for HPLC analysis every 1~3 h until the | Crystallization time | 6 h 47 min |
| difference between two consecutive samples of | The difference between two | 0.56% |
| intermediate B assay was ≤2.0% | consecutive samples of | |
| | intermediate B | |
| 23. Filtration | | |
| The mixture was filtered with stainless steel nutsche | n-Heptane | 35.1 kg |
| filter. The reactor wall or bottom was rinsed with n- | Area % of SME | ND |
| heptane (35.1 kg), and then the n-heptane rinsed the | | |
| filter cake. The filter cake was sampled for HPLC | | |
| analysis to make sure that the sample is free from | | |
| large quantity of impurities. | | |
| 24. Drying | | |
| The solid was dried at 15~25° C. 8 h later, the | Drying temperature | 15.0~24.6° C. |
| mixture was sampled for analysis every 4~8 h until | Drying time | 65 h 7 min |
| DCM residual ≤600 ppm; isopropyl acetate residual | DCM residual | 543 ppm |
| ≤5000 ppm; n-heptane residual ≤5000 ppm. During | Isopropyl acetate residual | Less than 3339 ppm |
| drying, the solid was turned over every 4~8 h. | n-Heptane residual | Less than 3339 ppm |

25. Product - Intermediate B − (+)-1-(2,4-difluoro-5-(2-(methylsulfinyl)ethoxy)phenyl)piperazine
Appearance: Off-white solid
Quantity: 29.5 kg, 28.7 kg corrected
Purity: 98.6%
Assay: 97.4%
Yield: 54.8%

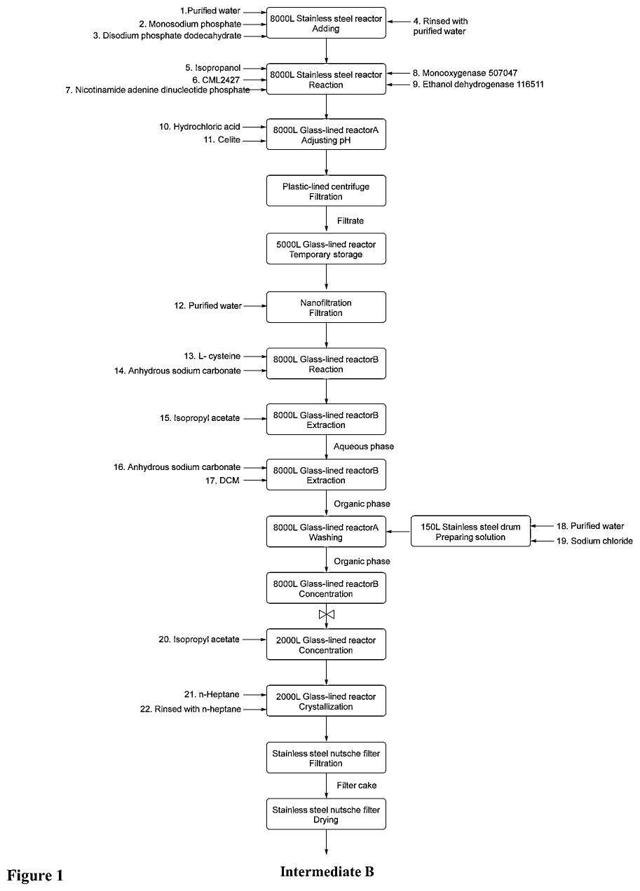

The detailed protocol is illustrated in FIG. 1. Materials used in the manufacturing process is summarized in Table 7.

TABLE 7

| | Mol. | Quantity | | Molar | Weight | |
|---|---|---|---|---|---|---|
| Material | Wt | (kg) | Mol. | Ratio | Ratio | Spec |
| SME | 288.4 | 49.7 | 172.3 | 1.0 | 1.0 | Identification: Conforms to |
| | | | | | | structure; Assay ≥97% |
| Monosodium | — | 82.8 | — | — | 1.7 | Characteristics: White solid; |
| phosphate | | | | | | Assay ≥98.0%; pH: 4.2~4.6; |
| | | | | | | Identification for Sodium: |
| | | | | | | Conforms; Identification for |
| | | | | | | phosphate: Conforms |
| Disodium phosphate | — | 3.4 | — | — | 0.1 | Characteristics: White solid; |
| dodecahydrate | | | | | | pH: 8.8~9.2; Identification for |
| | | | | | | Sodium: Conforms; |
| | | | | | | Identification for phosphate: |
| | | | | | | Conforms |
| Purified water | — | 3153.3 | — | — | 63.4 | Appearance: Clear and |
| | | | | | | colorless liquid and odorless; |
| | | | | | | Conductivity (25° C.): |
| | | | | | | Acceptance criteria <1.3 μs/cm; |
| | | | | | | TOC: Acceptance criteria |
| | | | | | | <500 ppbC; Microbial: Purified |
| | | | | | | water: Total microbial: |
| | | | | | | Acceptance |
| | | | | | | criteria <100 CFU/ml; |
| | | | | | | Objectionable Organism: |
| | | | | | | Should not be detected; test |
| | | | | | | methods refer to USP (Current |
| | | | | | | Version); Endotoxin: The |
| | | | | | | acceptance criterion is NMT 1 |
| | | | | | | EU/ml, test methods refer to |
| | | | | | | ChP (Current Version); Other: |
| | | | | | | Ammonia, pH, nitrate, nitrite, |
| | | | | | | and non-volatile substance |
| | | | | | | tests Ammonia, pH, nitrate, |
| | | | | | | nitrite, and non-volatile |
| | | | | | | substance tests: it should be |
| | | | | | | passed refers to testing |
| | | | | | | methods of CP (current |
| | | | | | | version) |
| Isopropanol | — | 78.7 | — | — | 1.6 | Characteristics: Colorless |
| | | | | | | liquid; Purity ≥99.0%; Water |
| | | | | | | content ≤0.2% w/w; |
| | | | | | | Identification: Conform with |
| | | | | | | standard spectrum |
| Nicotinamide | — | 1.0 | — | — | 0.02 | White to off-white solid; |
| adenine dinucleotide | | | | | | Assay ≥80%; Identification: |
| phosphate | | | | | | Conform to structure |
| Monooxygenase | — | 216.7 | — | — | 4 + 0.4 | Residue 1-(2,4-difluoro-5-(2- |
| Enzyme 3 | | | | | | (methylthio) ethoxy)phenyl) |
| | | | | | | piperazine of use-test ≤1.5% |
| Ethanol | — | 24.6 | — | — | 0.5 | Residue 1-(2,4-difluoro-5-(2- |
| dehydrogenase | | | | | | (methylthio) ethoxy)phenyl) |
| Enzyme 9 | | | | | | piperazine of use-test ≤1.5% |
| Hydrochloric acid | — | 88.4 | — | — | 1.8 | Characteristics: Colorless to |
| | | | | | | light yellow liquid; Assay: |
| | | | | | | ≥36% w/w |
| Celite | — | 25.0 | — | — | 0.5 | Exemption |
| L-Cysteine | — | 10.0 | — | — | 0.2 | White to off-white solid; |
| | | | | | | Assay ≥90%; Identification: |
| | | | | | | Conform to structure |
| Anhydrous sodium | 106.0 | 760.0 | 7169.8 | 41.6 | 15.3 | Characteristics: White solid; |
| carbonate | | | | | | Total alkali content (by |
| | | | | | | Na2CO3) ≥98.0%; |
| | | | | | | Identification for Sodium: |
| | | | | | | Conforms; Identification for |
| | | | | | | Carbonate: Conforms |
| Isopropyl acetate | — | 1047.6 | — | — | 21.0 | Characteristics: Colorless |
| | | | | | | liquid; Purity ≥99.0%; Water |
| | | | | | | content ≤0.1% w/w; |
| | | | | | | Identification: Conform with |
| | | | | | | standard spectrum |
| Dichloromethane | — | 1247.6 | — | — | 25.1 | Characteristics: Colorless |
| | | | | | | liquid; Purity ≥99.0%; Water |
| | | | | | | content ≤0.05%w/w; |
| | | | | | | Identification: Conform with |
| | | | | | | standard spectrum |
| n-Heptane | — | 117.0 | — | — | 2.4 | Colorless liquid, |
| | | | | | | Purity ≥97.0%, KF ≤0.1%, |
| | | | | | | Identification: Conform with |
| | | | | | | standard spectrum |

### 2.3 Synthesis of Intermediate (B) Using Conventional Synthetic Route

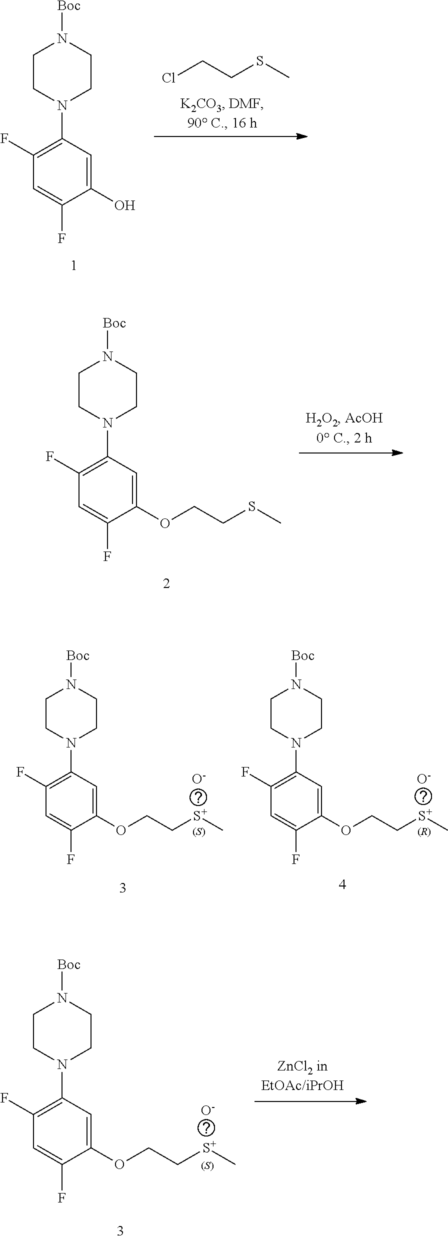

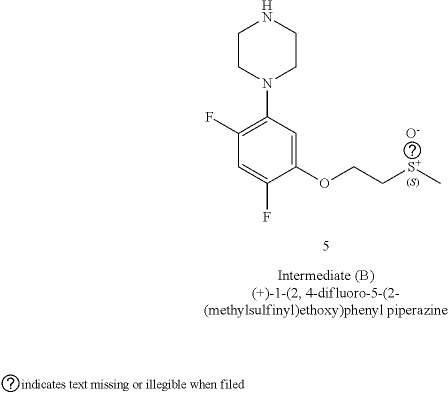

To a mixture of Compound 1 (60 kg, 191 mol, 1.0 eq) in acetonitrile (600 L) was added sodium hydroxide (22.9 kg, 573 mol) and 2-chloroethyl-methylsulfide (25.4 kg, 229 mol, 22.6 L) at 15° C. The mixture was stirred at 82° C. for 16 h. The reaction mixture was cooled to 15˜20° C. and then the reaction mixture was centrifuged and the filtrate was collected. The filter residue was stirred in ethylacetate (200 L) at 35˜40° C. for 2 h and then centrifuged and the filtrate was combined. The combined filtrate was decolorated with active carbon and filtered. The filter was concentrated until the residual solvent was 150 L. Then n-heptane (400 L) was added into the mixture and the mixture was concentrated until the solvent residual was 150 L. The residue was centrifuged and dried by drying oven to give Compound 2 (51 kg, 131 mol, 68.8% yield) as a light brown solid.

To a mixture of Compound 2 (50.5 kg, 130 mol, 1 eq) in acetic acid (200 L) was added aqueous hydrogen peroxide (17.7 kg, 156 mol, 15 L, 30% purity, 1.20 eq) at 10˜15° C. The mixture was stirred at 10˜15° C. for 3 h. The reaction was quenched with aq·Na₂S₂O₄ (25 Kg/200 L). The reaction mixture was then extracted with ethyl acetate (200 L×3). The pH value of the organic layer was adjusted to 8 by adding saturated aqueous sodium hydroxide (250 L). The combined organic phase was washed with brine (100 L) and concentrated at 45° C./−0.1 MPa until the residual solvent was 150 L. Then n-heptane (400 L) was added into the mixture and the mixture was concentrated until the solvent residual was 150 L. The residue was centrifuged and dried by drying oven to give a racemic mixture of enantiomers 3 and 4 (50.9 kg, 125 mol, 96.5% yield, 99.7% purity) as an off-white solid.

The racemic mixture of enantiomers 3 and 4 (29.52 Kg) was dissolved in methanol (628 L) and the coloured solution was decolorized with activated charcoal. The feed solution was then subjected to simulated moving bed chromatography, using as chiral stationary phase Chiralpak AD CSP (20 μm particle size, 1 Kg), packed in 8 columns at 5 cm ID and 10 cm length evenly. Methanol was used as mobile phase. Enantiomer 3 was collected as the first eluting enantiomer and the corresponding fractions were concentrated under reduced pressure to yield compound 3 (20.1 Kg, enantiomeric excess of 98.1% and 99.9% HPLC purity).

Compound 3 (9.06 Kg, 22.7 mol) was treated with zinc bromide (12.8 Kg; 56.8 mol), ethyl acetate (34.4 Kg) and isopropanol (7.4 Kg). The mixture is heated at 75-80° C. for 6 hours and after completion of the reaction, the reaction was cooled (precipitation) and aged. The crude product was filtered, washed with ethyl acetate and dried at ambient temperature. The crude was dissolved in a mixture of dichloromethane, 20 wt % aqueous sodium carbonate solution and 28-30 wt % aqueous ammonium hydroxide solution. The phases were allowed to separate, the lower organic layer containing the product was separated off and the aqueous layer was re-extracted with dichloromethane. The combined organic layers were concentrated by distillation at reflux, cooled, passed through a 1-micron filter then charged back to the reactor. The solution containing the product was solvent exchanged from dichloromethane to isopropyl acetate under reduced pressure and constant volume, cooled to ambient, n-heptane was added and the mixture cooled further to complete crystallization. The product Intermediate B was filtered, washed with n-heptane then dried (4.57 Kg, 67%).

### 2.4 Synthesis of A2A Inhibitors Through Coupling Reaction Between Intermediate (A) and Intermediate (B)

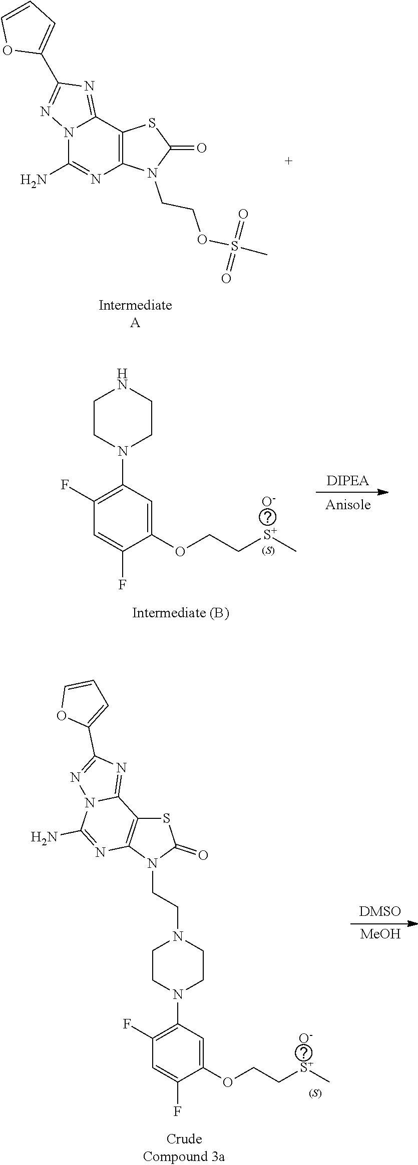

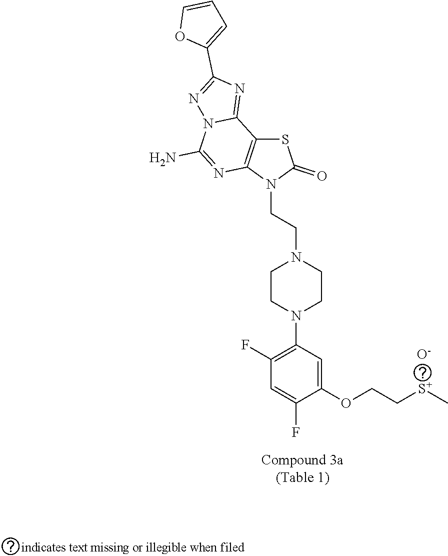

Intermediate A (23.2 Kg, 1.0 eq), Intermediate B (24.8 Kg, 1.4 eq) and anisole (93 Kg, 4.0 rel. vol) were added to the reactor and stirred. Then, N,N-diisopropylethylamine (11.5 Kg, 1.5 eq) and anisole (23.3 Kg, 1.0 rel. vol) were added and the reaction was heated to 105-115° C., and continued stirring for at least 20 hours. When reaction complete (confirmed by HPLC), the reactor contents was cooled to 70-80° C. Dose acetonitrile (128 Kg, 5.5 rel. vol) was added at 75±5° C. over at least 30 minutes, then it was cooled to 15-25° C. and stirred for 1 hour. The solids were isolated by filtration and washed with two portions of acetonitrile (each 55 Kg, 3.0 rel. vol), followed by two portions of methyl tert-butylether (each or 53 Kg, 3.0 rel. vol). The solids then were dried at 15-25° C. to give crude Compound 3A (29.1 Kg, 82.1% yield).

Crude compound 3a (22 Kg, 1.0 eq) and dimethyl sulfoxide (218 Kg, 9.0 rel. vol) were added to the reactor and stirred at 65-75° C. until fully dissolved. After 2 hours, the reaction was cooled to 45-50° C. and the contents were transferred through a polishing filter into a clean reactor, and stirred and heated to 55-65° C. Then, dose methanol (173 Kg, 10 rel. vol) whilst maintaining temp at 55-65° C. was added. After 2-4 hours stirring at 55-65° C., the reaction was cooled to 0±5° C. over a period of 3.5-4.5 hours, then stirred for a further 12-16 hours. The solids were isolated by filtration and washed twice with methanol (each 70 Kg, 4.0 rel. vol), then twice with methyl tert-butylether (each 65 Kg, 4.0 rel. vol). Finally, the solids were dried for at least 4 hours at 45-55° C. to yield Compound 3a (16.8 kg, 76% yield).

3. Enzymatic Biotransformation vs. Conventional Synthesis of Intermediate B

One of the important advantages of the enzymatic process is that it removes the necessity for a chiral separation using a chromatography on a chiral phase (SMB). Therefore, enzymatic biotransformation can produce the A2A inhibitors mentioned in this disclosure at a relatively higher yield in a cost-efficient way.

Conventional synthetic pathway for synthesis of Intermediate B showed that the SMB has a yield of approximately 40% (the other 40% is the other isolated enantiomer and 20% is lost as mixed fraction), whereas the enzymatic process has a yield of approximately 55% with high ee>99%.

There is also a clear cost advantage. For example, cost of chiral separation per kg of enantiomer produced by SMB is about 26,000 USD (based on 10 kg scale). On the other hand, cost of chiral oxidation per kg of enantiomer produced by enzymatic transformation is approximately 10,000 USD (based on 62 Kg scale). In addition, the cost per kg of the enzymatic process should decrease at higher scale, whereas the cost per kg of the SMB process does not really decrease much. A skilled person in the art knows that the cost of the processes can hugely vary depending on the various factors even global economy trends, however; costs calculated for the present disclosure should be evaluated under the time of filing.

Therefore, these two advantages mentioned above enhances industrial acceptance and suitability of the enzymatic transformation process of the present disclosure.

### 4. Sequences of the Lead Enzymes

TABLE 7

| | Accession | SEQ | |
|---|---|---|---|
| Enzyme | number | ID NO | Amino acid sequence |
| Monooxygenase | ABG97104.1 | 1 | MTTSMKAANPMNFPSTSDTGI |
| from | | | VDVLGVGAGFSGLYLSHRLTT |
| Rhodococcus | | | AGWTFAGFEAGPSVGGTWFW |
| jostii | | | NTYPGARCDVESIYYSYSFDEA |
| (Enzyme 3) | | | LQQEWTWSQRFAPQAEILSYIN |
| | | | HVADRFDLRKHFTFNTRVVGA |
| | | | TWNAAERLWEVQLDNGETRR |
| | | | GRYLISGAGGLSTPKDFDVPGL |
| | | | GNFTGLQVSTSRWNISLDDLA |
| | | | GKRVAVIGTGSSGVQAIPLIAE |
| | | | VAEHVTVFQRTPNYVMPARN |
| | | | AELPLERVDSIKDDYPAIREEC |
| | | | RHSPGGIPDRPVTDKAFDVSAE |
| | | | ERQRRYEAAYERSGFNGVGGE |
| | | | FADLLTDVEANRTASEFIHDKI |
| | | | REIVEDPATAELLVPRYHPLGA |
| | | | KRSVFGTDYYETYNRPNVSLV |
| | | | SLRDEPIETMTANAIVTSKGTY |
| | | | EADAVVLAIGFDAFTGPLYGL |
| | | | GLTGASGRKLQETWQDGIRTY |
| | | | LGMMTTDFPNFFMVAGPQSPA |
| | | | LASNVVMTIEQAVDWIADLIE |
| | | | HARDSGATLVEATPEGQNDW |
| | | | VDITEETVAQTLYATTDSWYR |
| | | | GSNVEGKPNTFMGYVGGVGK |
| | | | YRRMCTEIAKRGYPGVRIDGE |
| | | | TESPHLGPIHREIS |
| Ethanol | CAA46053.1 | 2 | MKGFAMLSIGKVGWIEKEKPAP |
| Dehydrogenase | | | GPFDAIVRPLAVAPCTSDIHTVFE |
| from | | | GAIGERHNMILGHEAVGEVVEV |
| Thermoanaerobium | | | GSEVKDFKPGDRVVVPAITPDWR |
| brockii | | | TSEVQRGYHQHSGGMLAGWKFS |
| | | | NVKDGVFGEFFHVNDADMNLA |
| | | | HLPKEIPLEAAVMIPDMMTTGFH |
| | | | GAELADIELGATVAVLGIGPVGL |
| | | | MAVAGAKLRGAGRIIAVGSRPV |
| | | | CVDAAKYYGATDIVNYKDGPIES |
| | | | QIMNLTEGKGVDAAIIAGGNADI |
| | | | MATAVKIVKPGGTIANVNYFGE |
| | | | GEVLPVPRLEWGCGMAHKTIKG |
| | | | GLCPGGRLRMERLIDLVFYKRVD |
| | | | PSKLVTHVFRGFDNIEKAFMLMK |
| | | | DKPKDLIKPVVILA |

### INCORPORATION BY REFERENCE

The entire disclosures of all patent and non-patent publications cited herein are each incorporated by reference in their entireties for all purposes.

### OTHER EMBODIMENTS

The disclosure set forth above may encompass multiple distinct disclosures with independent utility. Although each of these disclosures has been disclosed in its preferred form(s), the specific embodiments thereof as disclosed and illustrated herein are not to be considered in a limiting sense, because numerous variations are possible. The subject matter of the disclosures includes all novel and nonobvious combinations and subcombinations of the various elements, features, functions, and/or properties disclosed herein. The following claims particularly point out certain combinations and subcombinations regarded as novel and nonobvious. Disclosures embodied in other combinations and subcombinations of features, functions, elements, and/or properties may be claimed in this application, in applications claiming priority from this application, or in related applications. Such claims, whether directed to a different disclosure or to the same disclosure, and whether broader, narrower, equal, or different in scope in comparison to the original claims, also are regarded as included within the subject matter of the disclosures of the present disclosure.

What is claimed is:

1. A process for preparing (+)-1-(2,4-difluoro-5-(2-(methylsulfinyl)ethoxy)phenyl)piperazine (Intermediate B), or a pharmaceutically acceptable salt or solvate thereof, 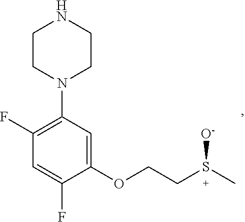
comprising the step of contacting 1-(2,4-difluoro-5-(2-(methylthio)ethoxy)phenyl)piperazine, or a pharmaceutically acceptable salt thereof: 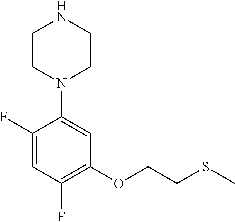 [SMILES: CSCCOc1cc(N2CCNCC2)c(F)cc1F]
with an enzyme in a solvent.

2. The process of claim 1, wherein the enzyme is a monooxygenase.

3. The process of any of claims 1-2, wherein the enzyme is enzyme 3 (Accession number: ABG97104.1).

4. The process of claims 1-3, wherein the step further comprises the addition of an additional enzyme.

5. The process of claim 4, wherein the additional enzyme is a crude alcohol dehydrogenase.

6. The process of claim 4-5, wherein the additional enzyme is a ketoreductase (KRED).

7. The process of any of claims 4-6, wherein the additional enzyme is Enzyme 9 (Accession number: CAA46053.1).

8. The process of any of claims 1-7, wherein the solvent comprises an alcohol.

9. The process of any of claims 1-8, wherein the solvent comprises isopropyl alcohol.

10. The process of any of claims 1-9, wherein the solvent comprises water.

11. The process of any of claims 1-10, wherein the process affords Intermediate B in at least 99% ee.

12. The process of any of claims 1-10, wherein the process affords Intermediate B in at least 99.9% ee.

13. A compound of formula Intermediate B: 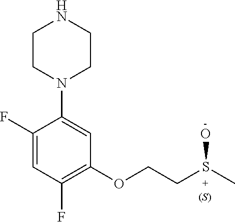
or a pharmaceutically acceptable salt thereof obtained by the step of contacting 1-(2,4-difluoro-5-(2-(methylthio)ethoxy)phenyl)piperazine, or a pharmaceutically acceptable salt thereof: 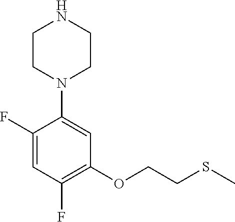 [SMILES: CSCCOc1cc(N2CCNCC2)c(F)cc1F]
with an enzyme in a solvent.

14. The compound of claim 13, wherein the enzyme is a monooxygenase.

15. The compound of any of claims 13-14, wherein the enzyme is enzyme 3 (Accession number: ABG97104.1).

16. The compound of any of claims 13-15, wherein the step further comprises the addition of an additional enzyme.

17. The compound of any of claims claims 13-16, wherein the additional enzyme is a ketoreductase (KRED).

18. The compound of any of claims 13-17, wherein the additional enzyme is Enzyme 9 (Accession number: CAA46053.1).

19. A process for preparing compund 3A 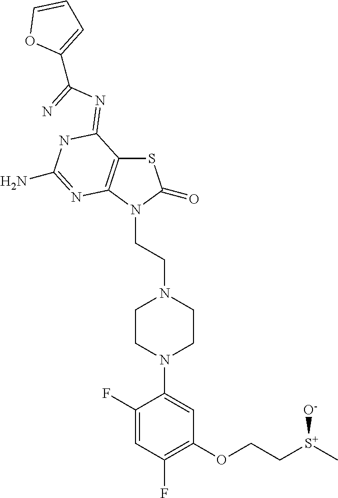 [SMILES: C[S@+]([O-])CCOc1cc(N2CCN(CCn3c(=O)sc4/c(=N/C(=N)c5ccco5)[nH]c(N)nc43)CC2)c(F)cc1F]
comprising the step of reacting Intermediate B with intermediate A: 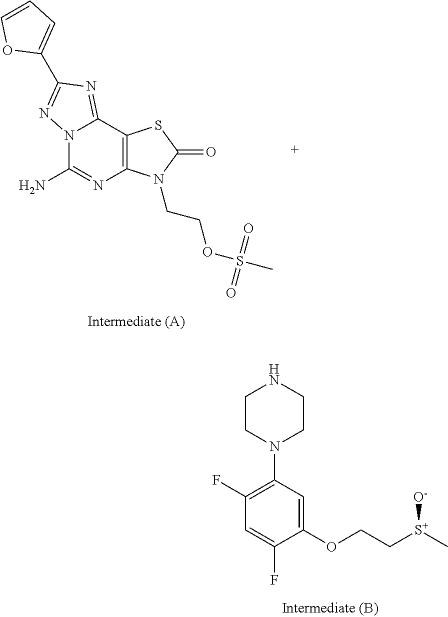 [SMILES: CS(=O)(=O)OCCn1c(=O)sc2c1nc(N)n1nc(-c3ccco3)nc21 ; C[S@+]([O-])CCOc1cc(N2CCNCC2)c(F)cc1F]
wherein Intermediate B was prepared using a process of any of claims 1-10.

20. The process of claim 19, wherein the step comprises addition of a base.

21. The process of claim 20, wherein the base is selected from the group of triethylamine (TEA), diisoproylethylamine (DIPEA), DEA, DIPA, and pyridine.

22. The process of claim 21, wherein the base is DIPEA.

23. The process of any of claim 19-22, wherein the step is performed in a solvent.

24. The process of claim 23, wherein the solvent is anisole.
